SEQUENCE LISTING (38 sequences)

SEQ ID NO: 1 - protein, 25 aa, Artificial Sequence
  Synthetic
ALLTSGTSDPRARVTYGTPSLWARL

SEQ ID NO: 2 - protein, 25 aa, Artificial Sequence
  Synthetic
MSTEEARFLTYHMWPLTFLSPSELA

SEQ ID NO: 3 - protein, 25 aa, Artificial Sequence
  Synthetic
FQQLTCVLPILDNLLKANVINKQEH

SEQ ID NO: 4 - protein, 25 aa, Artificial Sequence
  Synthetic
ALSTSSPIPSVPQSQAQPQTGSGAS

SEQ ID NO: 5 - protein, 25 aa, Artificial Sequence
  Synthetic
SVPQSQAQPQTGSGASRALPSWQEV

SEQ ID NO: 6 - protein, 24 aa, Artificial Sequence
  Synthetic
KDQRRNVGNMQPTAQYSGSSTISL

SEQ ID NO: 7 - protein, 25 aa, Artificial Sequence
  Synthetic
PNQSSRAFQGTDHSSDLAFDFLSQQ

SEQ ID NO: 8 - protein, 25 aa, Artificial Sequence
  Synthetic
EMNSVKLRSVKRSEQDVKPKPVDAT

SEQ ID NO: 9 - protein, 25 aa, Artificial Sequence
  Synthetic
NHSQVVQPVTTTGPEHSKPLEKSDG

SEQ ID NO: 10 - protein, 25 aa, Artificial Sequence
  Synthetic
DVDKLREQLSTSENALTGRILDLKT

SEQ ID NO: 11 - protein, 25 aa, Artificial Sequence
  Synthetic
KADGIMWLAKACWSIQSEMPCIQAQ

SEQ ID NO: 12 - protein, 25 aa, Artificial Sequence
  Synthetic
GVRTCEGCKGFFKRTVQKNAKYICL

SEQ ID NO: 13 - protein, 23 aa, Artificial Sequence
  Synthetic
EMEASTYDGFIWKISDFARKRQE

SEQ ID NO: 14 - protein, 25 aa, Artificial Sequence
  Synthetic
RTREASENSGTRSRAWLAVALGAGG

SEQ ID NO: 15 - protein, 4 aa, Artificial Sequence
  Synthetic
  MISC_FEATURE (1)..(1): D-amino acid
  MISC_FEATURE (2)..(2): 2,6-dimethyl-L-tyrosine
  MISC_FEATURE (3)..(3): Ornithine
RXXF

SEQ ID NO: 16 - protein, 4 aa, Artificial Sequence
  Synthetic
  MISC_FEATURE (2)..(2): D-amino acid
RRRK

SEQ ID NO: 17 - protein, 29 aa, Artificial Sequence
  Synthetic
  MISC_FEATURE (26)..(26): D-amino acid
  MISC_FEATURE (27)..(27): 2,6-dimethyl-L-tyrosine
  MISC_FEATURE (28)..(29): Ornithine
ALSTSSPIPSVPQSQAQPQTGSGASRXXF

SEQ ID NO: 18 - protein, 29 aa, Artificial Sequence
  Synthetic
  MISC_FEATURE (1)..(1): D-amino acid
  MISC_FEATURE (2)..(2): 2,6-dimethyl-L-tyrosine
  MISC_FEATURE (3)..(3): Ornithine
RXXFALSTSSPIPSVPQSQAQPQTGSGAS

SEQ ID NO: 19 - protein, 29 aa, Artificial Sequence
  Synthetic
  MISC_FEATURE (2)..(2): D-amino acid
RRRKALSTSSPIPSVPQSQAQPQTGSGAS

SEQ ID NO: 20 - protein, 29 aa, Artificial Sequence
  Synthetic
  MISC_FEATURE (27)..(27): D-amino acid
ALSTSSPIPSVPQSQAQPQTGSGASRRRK

SEQ ID NO: 21 - protein, 29 aa, Artificial Sequence
  Synthetic
  MISC_FEATURE (26)..(26): D-amino acid
  MISC_FEATURE (27)..(27): 2,6-dimethyl-L-tyrosine
  MISC_FEATURE (28)..(28): Ornithine
PNQSSRAFQGTDHSSDLAFDFLSQQRXXF

SEQ ID NO: 22 - protein, 29 aa, Artificial Sequence
  Synthetic
  MISC_FEATURE (1)..(1): D-amino acid
  MISC_FEATURE (2)..(2): 2,6-dimethyl-L-tyrosine
  MISC_FEATURE (3)..(3): Ornithine
RXXFPNQSSRAFQGTDHSSDLAFDFLSQQ

SEQ ID NO: 23 - protein, 29 aa, Artificial Sequence
  Synthetic
  MISC_FEATURE (2)..(2): D-amino acid
RRRKPNQSSRAFQGTDHSSDLAFDFLSQQ

SEQ ID NO: 24 - protein, 29 aa, Artificial Sequence
  Synthetic
  MISC_FEATURE (27)..(27): D-amino acid
PNQSSRAFQGTDHSSDLAFDFLSQQRRRK

SEQ ID NO: 25 - protein, 29 aa, Artificial Sequence
  Synthetic
  MISC_FEATURE (26)..(26): D-amino acid
  MISC_FEATURE (27)..(27): 2,6-dimethyl-L-tyrosine
  MISC_FEATURE (28)..(28): Ornithine
ALLTSGTSDPRARVTYGTPSLWARLRXXF

SEQ ID NO: 26 - protein, 29 aa, Artificial Sequence
  Synthetic
  MISC_FEATURE (1)..(1): D-amino acid
  MISC_FEATURE (2)..(2): 2,6-dimethyl-L-tyrosine
  MISC_FEATURE (3)..(3): Ornithine
RXXFALLTSGTSDPRARVTYGTPSLWARL

SEQ ID NO: 27 - protein, 29 aa, Artificial Sequence
  Synthetic
  MISC_FEATURE (2)..(2): D-amino acid
RRRKALLTSGTSDPRARVTYGTPSLWARL

SEQ ID NO: 28 - protein, 29 aa, Artificial Sequence
  Synthetic
  MISC_FEATURE (27)..(27): D-amino acid
ALLTSGTSDPRARVTYGTPSLWARLRRRK

SEQ ID NO: 29 - protein, 29 aa, Artificial Sequence
  Synthetic
  MISC_FEATURE (1)..(1): D-amino acid
  MISC_FEATURE (2)..(2): 2,6-dimethyl-L-tyrosine
  MISC_FEATURE (3)..(3): Ornithine
RXXFFQQLTCVLPILDNLLKANVINKQEH

SEQ ID NO: 30 - protein, 29 aa, Artificial Sequence
  Synthetic
  MISC_FEATURE (26)..(26): D-amino acid
  MISC_FEATURE (27)..(27): 2,6-dimethyl-L-tyrosine
  MISC_FEATURE (28)..(28): Ornithine
FQQLTCVLPILDNLLKANVINKQEHRXXF

SEQ ID NO: 31 - protein, 29 aa, Artificial Sequence
  Synthetic
  MISC_FEATURE (2)..(2): D-amino acid
RRRKFQQLTCVLPILDNLLKANVINKQEH

SEQ ID NO: 32 - protein, 326 aa, Homo sapiens [sequence omitted]

SEQ ID NO: 33 - DNA, 23 bp, Artificial Sequence
  Synthetic
  misc_feature: siRNA
aagcgguguuucucagaattgtt

SEQ ID NO: 34 - DNA, 22 bp, Artificial Sequence
  Synthetic
  misc_feature: siRNA
aacaauucugagaaacccgctt

SEQ ID NO: 35 - protein, 25 aa, Artificial Sequence
  Synthetic
  MISC_FEATURE (1)..(25): D-amino acid
ALLTSGTSDPRARVTYGTPSLWARL

SEQ ID NO: 36 - protein, 7 aa, Artificial Sequence
  Synthetic
HAIYPRH

SEQ ID NO: 37 - protein, 16 aa, Artificial Sequence
  Synthetic
RQIKIWFQNRRMKWKK

SEQ ID NO: 38 - protein, 29 aa, Artificial Sequence
  Synthetic
  MISC_FEATURE (1)..(26): D-amino acid
  MISC_FEATURE (27)..(27): 2,6-dimethyl-L-tyrosine
  MISC_FEATURE (28)..(28): Ornithine
ALLTSGTSDPRARVTYGTPSLWARLRXXF

(19) United States
(12) Patent Application Publication
(10) Pub. No.: US 2025/0051409 A1
(43) Pub. Date: Feb. 13, 2025

(54) MODULATORS OF PHOSPHATIDYLSERlNE DECARBOXYLASE AND USE THEREOF

(71) Applicant: THE NATIONAL INSTITUTE FOR BIOTECHNOLOGY IN THE NEGEV LTD., Beer Sheva (IL)

(72) Inventor: Varda SHOSHAN-BARMATZ, Omer (IL)

(21) Appl. No.: 18/548,600

(22) PCT Filed: Mar. 2, 2022
(86) PCT No.: PCT/IL2022/050230

§ 371 Date: Sep. 1, 2023

Related U.S. Application Data
(60) Provisional application No. 63/155,819, filed on Mar. 3, 2021.

(51) Int. Cl. C07K 14/47; A61K 38/00; A61P 35/00

(52) U.S. Cl. CPC C07K 14/4703; A61P 35/00; A61K 38/00

(57) ABSTRACT

The present invention relates to compositions and methods for treating cancer, particularly to agents that capable of modulating the activity of phosphatidylserine decarboxylase (PSD) within a mammalian cell, particularly to peptides modulating its activity in the mitochondria and the nucleus, useful for treating cancer diseases.

The Sequence Listing in ASCII text file format of 17,579 bytes in size, created on Feb. 26, 2024, with the file name “2024-02-26SequenceListing_SHOSHAN13_ST25,” filed in the U.S. Patent and Trademark Office on even date herewith, is hereby incorporated herein by reference.

### FIELD OF THE INVENTION

The present invention relates to compositions and methods for treating cancer, particularly to agents that bind to phosphatidylserine decarboxylase (PSD) within mammalian cells and modulate its activity, particularly to peptides binding to and modulating PSD activity, useful for treating cancer diseases.

### BACKGROUND OF THE INVENTION

Alteration of tumor metabolism, including lipid metabolism is involved in carcinogenesis, with cancer cells also demonstrating a high dependence on lipids (Baenke F, et al., 2013. Dis Model Mech 6, 1353-1363). Phospholipids (PLs) are not only essential building-block components of cellular membranes, but act as regulators for various cellular functions such as cell adhesion and migration, neurotransmission, signal transduction, vesicular trafficking, apoptosis, metabolism, and post-translational modifications (Shevchenko A, et al., 2010. Nat Rev Mol Cell Biol 11, 593-598).

Changes in composition, distribution, and metabolism of PLs in cells, tissues, and body fluids (blood, urine) are associated with cancer and other diseases (Adibhatla R M, et al., 2006. AAPS J 8, E314-321). Aberrant phospholipid metabolism in cancer has recently been established as a universal metabolic hallmark of cancer, and the phospholipid content was shown to increase with cell transformation and tumor progression (Dobrzynska I, et al., 2015. J Membr Biol 248, 301-307). Changes in PL profiles have been associated with cancer diagnosis and treatment and specific lipids might be involved in the onset and evolution of cancer (Wenk M R, et al., 2005. Nat Rev Drug Discov 4, 594-610; Bandu R, et al., 2018. Mass Spectrom Rev 37, 107-138).

Phosphatidylethanolamine (PE) is the second most abundant phospholipid on mammalian cellular membranes. Comprising ˜25% of mammalian phospholipids, it is found predominantly in the inner leaflet of the plasma membrane and enriched in mitochondrial inner membranes (Vance J E, and Tasseva G, 2013. Biochim Biophys Acta 1831, 543-554). Beside functioning as a membrane structural element, PE participates in many important pathophysiological cellular processes (Calzada E, et al., 2016. Int Rev Cell Mol Biol 321, 29-88). It has been demonstrated that translocation and redistribution of PE occurs during cell division and cell death, and that it is important for membrane fusion and remodeling. PE itself or PE-derived ethanolamine is covalently linked to diverse proteins, including signaling proteins, and lipidation of ubiquitin-like protein LC3 by PE is a prerequisite for autophagosome formation (Vance et al., 2013, ibid). PE synthesized in mitochondria was found to be important for mitochondrial function (Tasseva G, et al., 2013. J Biol Chem 288, 4158-4173). PE is long known to be elevated in several cancers (Podo F. 1999. Tumor phospholipid metabolism. NMR Biomed 12, 413-439).

In mammalian cells, distinct pools of PE are synthesized in either the mitochondria (Percy A K, et al., 1983. Arch Biochem Biophys 223, 484-494) or in the endoplasmic reticulum (ER) membranes (Vance et al., 2013, ibid). Mitochondrial-associated membranes (MAM) act as bridges between the mitochondria and endoplasmic reticulum (ER), with versatile functions, including support of mitochondrial function and the synthesis of neutral lipids as well as phospholipids (Vance J E. 2014. Biochim Biophys Acta 1841, 595-609; Rusinol A E, et al., 1994. J Biol Chem 269, 27494-27502). In the MAM, phosphatidylserine (PS) is transferred to mitochondria (Achleitner G, et al., 1999. Eur J Biochem 264, 545-553) and is converted to PE by the mitochondrial enzyme phosphatidylserine decarboxylase (PSD) (Di Bartolomeo F, et al., 2017. Biochim Biophys Acta Mol Cell Biol Lipids 1862, 25-38). Loss of PSD causes defects in mitochondrial morphology and function, and disrupts electron transport chain (ETC) complex formation (Vance et al., 2013, ibid). PSD is an essential protein with its knock out (KO) being embryonic lethal (Steenbergen R, et al., 2005. J Biol Chem 280, 40032-40040).

SMAC/Diablo (second mitochondria-derived activator of caspase/direct inhibitor of apoptosis-binding protein with low pI) is a pro-apoptotic mitochondrial intermembrane space (IMS) protein (Verhagen A M, et al., 2000. Cell 102, 43-53; Du C, et al., 2000. Cell 102, 33-42). It was, however, described that SMAC is overexpressed in cancer cells (Yoo N J, et al. 2003. APMIS 111, 382-388; Kempkensteffen C, et al., 2008. J Cancer Res Clin Oncol 134, 543-550; Paul A, et al., 2018. Mol Ther 26, 680-694). The Inventor of the present invention and co-workers have identified a new non-apoptotic function of SMAC/Diablo of being associated with regulating lipid synthesis essential for cancer growth and development (Paul A, et al., 2018. ibid; International (PCT) Application publication No. WO 2019/021289). It was demonstrated that silencing SMAC expression using human specific siRNA (si-hSMAC) decreased PLs levels including phosphatidylcholine (PC) levels along with alterations in the expression levels of enzymes associated with their synthesis. In addition, SMAC/Diablo depletion led to a decrease in vesicle formation, inhibition of cell proliferation in cancer cell lines and reduced growth of lung cancer cell-derived tumor. SMAC depletion also alters nuclear morphology, and the expression of genes associated with the cell membrane, exosomes, and ER- and Golgi-related proteins.

Publication of the inventor of the present invention and co-workers, published after the priority date of the present invention, describes six nuclear proteins ARNT, BIRC2, MAML2, NR4A1, BIRC5 and HTRA2, five of which also interacted with PSD through sequences that are not involved in SMAC binding. Synthetic peptides carrying the PSD-interacting sequence could bind purified PSD and inhibit the PSD catalytic activity. When targeted specifically to the mitochondria or the nucleus, these cell-penetrating synthetic peptides inhibited cancer cell proliferation and tumor growth in mouse models.

Given that fundamental differences exist between the cellular membranes of healthy cells and tumor cells, and the signaling function of PE, lipids in the cellular membrane may be a potential target for cancer therapy.

There is a need for and it would be highly advantageous to have composition of methods for targeting phospholipid-associated processes essential for cancer development and progression for treating cancer diseases.

### SUMMARY OF THE INVENTION

The present invention answers the above-described needs, providing compositions and method for treating various cancer diseases via intervention in the biosynthesis of cellular phospholipids, leading to inhibition of cancer cell proliferation and tumor growth.

The present invention is based in part on the unexpected finding that proteins interacting with SMAC/Diablo also directly and independently bind to PSD, and further on the finding that the identified PSD-binding sites in these proteins, in form of peptides, also bind to purified PSD. These peptides, when targeted to the mitochondrion or the nucleus of a mammalian cell, inhibits PSD activity, resulting in inhibition of cancer cell proliferation and tumor growth in mice model. Without wishing to be bound by any specific theory or mechanism of action, the binding of the peptide to PSD within the mammalian cell may result in a decrease of the phospholipid content within the cell, leading to dysfunction or disruption of the cell and organelle membranes and to inhibition of cell growth and proliferation, and to cellular anti-inflammation activity.

According to certain aspects, the present invention provides an agent capable of modulating the activity of a phosphatidylserine decarboxylase (PSD) within a mammalian cell.

According to certain embodiments, the agent capable of modulating the activity of PSD is selected from the group consisting of a peptide, a small molecule, a nucleic acid molecule and any combination thereof.

According to certain embodiments, the agent is capable of interacting with PSD.

According to certain exemplary embodiments, the agent is capable of inhibiting the activity of PSD.

According to certain exemplary embodiments, the agent is a peptide. According to some embodiments, the peptide is a synthetic or recombinant peptide.

According to some embodiments, the peptide modulating the activity of PSD is derived from a polypeptide or protein interacting with PSD.

According to certain exemplary embodiments, the protein interacting with PSD is selected from the group consisting of MAML2, HTRA2, BIRC2, TRAF-2, MTFR1, ARNT, and any combination thereof. Each possibility represents a separate embodiment of the present invention.

According to certain embodiments, the peptide modulating the activity of PSD comprises an amino acid sequence having at least 80% identity over at least 23 contiguous amino acids of a sequence selected from the group consisting of SEQ ID NOs:1, 4, 5, 2, 6-9, and 11-14, an analog, derivative or a fragment thereof. Each possibility represents a separate embodiment of the present invention.

According to certain embodiments, the peptide modulating the activity of PSD comprises an amino acid sequence selected from the group consisting of SEQ ID NOs:1, 4, 5, 2, 6-9, and 11-14, an analog, derivative or a fragment thereof. According to certain exemplary embodiments, the peptide modulating the activity of PSD consists of the amino acid sequence set forth in any one of SEQ ID NOs:1, 4, 5, 2, 6-9, and 11-14. Each possibility represents a separate embodiment of the present invention.

According to certain exemplary embodiments, the peptide modulating the activity of PSD, designated herein IC11, comprises an amino acid sequence having at least 80% identity over at least 23 contiguous amino acids of the amino acid sequence set forth in SEQ ID NO:1. According to some embodiments, the peptide modulating the activity of PSD comprises the amino acid sequence set forth in SEQ ID NO:1. According to some embodiments, the peptide modulating the activity of PSD consists of the amino acid sequence set forth in SEQ ID NO:1.

According to certain further exemplary embodiments, the peptide modulating the activity of PSD comprises an amino acid sequence having at least 80% identity over at least 23 contiguous amino acids of a sequence set forth in SEQ ID NO:4. According to some embodiments, the peptide modulating the activity of PSD comprises the amino acid sequence set forth in SEQ ID NO:4. According to some embodiments, the peptide modulating the activity of PSD consists of the amino acid sequence set forth in SEQ ID NO:4.

According to certain embodiments, the peptide modulating the activity of PSD further comprises at least one nuclear and/or mitochondrial targeting moiety.

The mitochondrial and/or nuclear targeting moiety can be peptidic or non-peptidic, and is covalently linked to the peptide modulating PSD activity directly or a via linker. According to certain exemplary embodiments, the targeting moiety is a peptide. According to theses embodiments, the targeting moiety may be linked to the peptide at any position, as long as the resulting peptide preserves the PSD modulating activity. According to certain exemplary embodiments, the targeting moiety is linked to the N- or the C-terminus of the peptide modulating the activity of PSD. When the peptide comprises a combination of mitochondrial and nuclear targeting moieties, each moiety may be independently linked to the N- or C-terminus of the peptide or the mitochondrial and nuclear targeting moieties can be linked in tandem to the N- or C-terminus of the peptide.

Any mitochondrial and/or nuclear targeting moiety can be used according to the teachings of the present invention. According to certain exemplary embodiments, the mitochondrial targeting moiety comprises the amino acid sequence D-Arg-Dmt-Orn-Phe-NH₂, where Dmt is 2,6-dimethyl-L-tyrosine (SEQ ID NO:15). According to certain exemplary embodiments, the nuclear targeting moiety is the tetrapeptide RrRK, wherein r is D-arginine (SEQ ID NO:16).

According to certain exemplary embodiments, the peptide modulating the activity of PSD further comprises a mitochondrial targeting moiety comprising an amino acid sequence selected from the group consisting of SEQ ID NO:17, SEQ ID NO:21, and SEQ ID NO:25. Each possibility represents a separate embodiment of the present invention.

According to certain further exemplary embodiments, the peptide modulating the activity of PSD further comprises a nuclear targeting moiety comprising an amino acid sequence selected from the group consisting of SEQ ID NO:19, SEQ ID NO:20, SEQ ID NO:24 and SEQ ID NO:28. Each possibility represents a separate embodiment of the present invention.

According to some embodiments, the peptide modulating PSD activity further comprises a cell penetration moiety enhancing the permeability of the peptide through the cell plasma membrane. Any cell penetrating moiety as is known in the art can be used according to the teachings of the present invention. According to certain exemplary embodiments, the cell penetration moiety is a peptide (cell penetrating peptide, CPP).

According to certain embodiments, the moiety targeting the inhibitory peptide to the mitochondrion or to the nucleus further enhances the peptide permeability through the cell plasma membrane (further serving as cell penetration moiety).

The peptides modulating PSD activity, and/or the mitochondrial or nuclear targeting peptides and/or the CPPs according to the teachings of the present invention can comprise amino acids in L-configuration, D-configuration or a combination thereof. Each possibility represents a separate embodiment of the present invention.

According to certain embodiments, the agent capable of modulating the activity of PSD, inhibits its activity.

According to certain embodiments, the agent capable of modulating the activity of PSD, particularly a peptide comprising a mitochondrial and/or nuclear targeting peptide, when present within the cell, is capable of inhibiting cell proliferation.

According to some embodiments, the agent capable of modulating the activity of PSD, particularly a peptide comprising a mitochondrial and/or nuclear targeting peptide, when present in a cell, is capable of inhibiting phosphatidylethanolamine (PE) synthesis within the cell.

According to certain embodiments, the cell is a cancerous cell.

According to certain additional aspects, the present invention provides a pharmaceutical composition comprising a therapeutically effective amount of at least one agent capable of modulating the activity of PSD.

According to certain exemplary embodiments, the pharmaceutical composition further comprises at least one pharmaceutically acceptable diluent, excipient or carrier.

According to certain embodiments, the at least one agent capable of modulating the activity of PSD is selected from the group consisting of a peptide, a small molecule, a nucleic acid molecule and any combination thereof. According to certain embodiments, the agent is capable of modulating the activity of PSD in a mammalian cell, particularly human cell. According to certain exemplary embodiments, the mammalian cell is a cancerous cell.

According to certain embodiments, the at least one agent is capable of interacting with PSD. According to certain exemplary embodiments, the agent is capable of inhibiting the activity of PSD.

According to certain exemplary embodiments, the at least one agent is a peptide. The at least one peptide is as described hereinabove.

According to yet certain further aspects, the present invention provides a method for treating cancer in a subject in need thereof, comprising administering to the subject a therapeutically effective amount of at least one agent capable of modulating the activity of phosphatidylserine decarboxylase (PSD) or a pharmaceutical composition comprising same.

According to certain embodiments, the at least one agent modulates the activity of PSD in cancerous cells of the subject. According to some embodiments, the subject is a mammal. According to certain exemplary embodiments, the subject is a human.

According to certain embodiments, the at least one agent is selected from the group consisting of a peptide, small molecule, a nucleic acid molecule and any combination thereof.

According to certain embodiments, the at least one agent is capable of interacting with PSD within the cells of the subject. According to certain exemplary embodiments, the agent is capable of inhibiting the activity of PSD.

According to certain exemplary embodiments, the agent is a peptide.

The at least one peptide is as described hereinabove.

According to certain embodiments, modulating the activity of PSD within cancerous cells of the subject results in reduced proliferation of the cancerous cells.

Unexpectedly, when administered intravenously to a subject, the at least one agent, particularly a peptide comprising nuclear or mitochondrial targeting moiety does not negatively affect non-cancerous cells of the subject.

According to certain embodiments, the cancer cells overexpress PSD compared to corresponding healthy cells.

According to some embodiments, overexpression is mRNA overexpression. According to some embodiments, overexpression is protein overexpression. According to some embodiments, the cancer cells comprise a significantly higher expression of PSD as compared to corresponding healthy cells. For example, the cancer cells express 1.5 or 2-fold higher levels of PSD as compared to corresponding healthy cells.

According to certain embodiments, the cancer is selected from the group consisting of lung cancer, breast cancer, colon cancer, esophagus cancer, lymphoma, sarcoma, stomach cancer, skin cancer, renal cancer, prostate cancer, testicular cancer, cervical cancer, ovarian cancer, leukemia and pancreatic cancer. Each possibility represents a separate embodiment of the present invention.

According to certain currently exemplary embodiments, the cancer is lung cancer.

According to certain currently exemplary embodiments, the cancer is breast cancer

According to certain embodiments, treating the cancer comprises re-programming the cancerous cells to at least one of normal phospholipid synthesis and decreased proliferation.

Any method as is known in the art for administering the agent that modulates the activity of PSD can be used according to the teachings of the present invention. According to some embodiment, the agent is administered within a pharmaceutical composition. According to certain exemplary embodiments, the pharmaceutical composition further comprises pharmaceutically acceptable excipients, diluents or carriers.

According to certain embodiments, the agent modulating the activity of PSD is a peptide.

According to certain embodiments, the agent modulating the activity of PSD or a pharmaceutical composition comprising same is administered via intravenous, intradermal, intramuscular, intra-arterial, intralesional, percutaneous, subcutaneous, intranasal or oral administration or by inhalation or by aerosol administration, or by combinations thereof. In some embodiments, administration is prophylactic administration, and in alternative embodiments, administration is therapeutic administration. Each possibility represents a separate embodiment of the present invention.

The mode of administering the at least one agent according to the teachings of the present invention will depend upon the type of the agent, the type and severity of the cancer and parameters related to the subject (age, gender, weight etc.).

According to certain embodiments, the method of the present invention further comprises administering to the subject at least one additional anti-cancer agent or anti-cancer therapy.

According to certain embodiments, the at least one additional anti-cancer agent is an inhibitor of SMAC/Diablo expression and/or activity. The SMAC/Diablo inhibitor may be, for example, an RNA inhibitory molecule or a peptide as described in WO 2019/021289.

According to additional certain aspects, the present invention provides use of at least one agent that modulates the activity of PSD or a pharmaceutical composition comprising same for treating cancer.

The at least one agent, the pharmaceutical composition and the cancer types are as described hereinabove.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 demonstrates that si-hSMAC-A treatment attenuates growth of A549 lung cancer tumor xenograft and altered phospholipid levels. (A) Tumor growth of the A549 cell xenograft in immunodeficient 6-week-old male athymic nude mice. Mice were treated twice a week intratumourally with si-hSMAC-A (350 and 700 nM) or with non-targeted si-RNA (si-NT, 350 nM) (n=8 mice/group). Treatment was initiated when the average tumor volume in each group reached ˜50 mm³. Results are presented as the mean tumor volume±SEM. (B, C) Immunoblot (B) and quantitative analysis (C) of SMAC, VDAC1 and citrate synthase (CS) in si-hSMAC-TTs and si-NT-TTs samples (n=6 mice/group). (D-F) IHC stained of si-hSMAC-TTs or si-NT-TTs sections for SMAC (D), Ki-67 (E), and their quantitative analysis (F) (n=6 mice/group). Scale bars represent 50, 25 or 15 μm as indicated. (G) PL, PC and PE analysis was carried out as described in the Materials and methods section hereinbelow (n=6 mice per group). Results are the means±SEM. P values were calculated using two-sided Student's t-test, **P≤0.01; ***P 0.001.

FIG. 2 demonstrates that SMAC knockout in A549 cells by CRISPR/Cas inhibits cell proliferation that could be restored upon SMAC re-expression. (A) Schematic presentation of CRISPR/Cas9 mediates knockout. (B) immunoblotting stained with anti-SMAC antibodies and Quantification (RU, relative units) of SMAC in control and CRISPR/Cas9-generated SMAC-deficient A549 cells and HEK-293T cells (C). Cell proliferation in SMAC knockout A549 and HEK-293T cells (n−4) as analyzed using the SRB method (n=4). (D, E) A549 cells expressing SMAC were transfected with control plasmid, and SMAC knockout A549 cells were transfected with plasmid pCDNA3.1 (0.5 or 1 μg DNA) encoding full-length SMAC. After 48 h cells were analyzed by immunoblotting for SMAC expression (D) and cell proliferation (n=4) (E). Results are the means_SEM, P values were calculated using two-sided Student's t-test, **P≤0.01; ***P≤0.001; NS, nonsignificant.

FIG. 3 shows subcellular morphological alterations including ER-mitochondria contact sites, as induced by reduction of SMAC levels. (A, B) Representative transmission electron micrographs of sections from si-NT-TTs (A) and si-hSMAC-A-TTs (B) from A549 xenografts carried out as described previously (Paul et al., 2021, ibid). Various membrane organelles as intracellular vesicles containing surfactant-accumulating lamellar bodies MAM-like structures (ER-associated mitochondria) are seen in the si-NT-TTs (Aa-c), but not in si-hSMAC-A-TTs, showing enrichment in the mitochondria (m), ER and nucleus (Nu). Arrows (b) and (e, f) point to lamellar bodies (A b) and ER (B e, J), respectively. Mitochondria number in si-NT-TTs and si-hSMAC-A-TTs per EM section was 1-3 and 8-15, respectively. Scale bars represent 2 or 0.5 μm as indicated. (C) IF staining for IP3R and VDAC1 in si-NT-TTs and si-hSMAC-A-TTs sections. Images were captured by confocal microscope, subjected to quantitative analysis (n=5). (D) si-NT-TTs and si-hSMAC-TTs were subjected to in situ PLA to test for close association between VDAC1 (OMM) and IP3R (ER) (MAM) using specific antibodies. Ligation product was subjected to quantification. Results are the means±SEM, P values were calculated using two-sided Student's t-test, ***P≤0.001.

FIG. 4 demonstrates that SMAC knockout in A549, but not in HEK-293T cells reduces PL and PC, and increases PE. (A) Schematic presentation of PE reaction with DSB-3 and obtained fluorescence signals used for PSD activity analysis. (B) PLs were extracted from the indicated cells and analyzed for total PL, PC and PE levels as described in the Materials and Methods section hereinbelow. (C) Schematic presentation of PS imported from the ER into the mitochondria at the MAM and its conversion to PE by PSD. PSD activity (D) and PE levels (E) in control and SMAC-KO, A549 and HEK-293T cells analyzed as described in the Materials and Methods section hereinbelow. Control and SMAC-knock out A549 cells and their mitochondria free and mitochondria-enriched subfractions were subjected to immunoblotting (F) or PSD activity assay (G). Total PL, PC and PE as analyzed in SMAC-KO A549 cell extract, mitochondria-free and mitochondria-enriched fractions relative to their levels in SMAC-expressing A549 cells (H). Results are the mean±SEM (n=3); P values were calculated using two-sided Student's t-test, *P≤0.05, **P≤0.01; ***P≤0.001, NS, nonsignificant.

FIG. 5 shows confocal fluorescence imaging of PE as stained with DSB-3. A549 cells were stained with MitoTracker (250 nM) for 45 min followed by incubation with DSB-3 (10 μM) for 2 h. Nuclei were stained with DAPI. (A, B) Representative images showing DSB-3-labeled PE at the plasma membrane and in the mitochondria. (C) Magnification of the squared sections in (A, B) to point to plasma membranes (arrows, A a, c, d), mitochondria (arrows within squares, b, c, e), and apoptotic cell with blubbing membrane is shown in C, e. (D) Control and SMAC-OK A549 cells stained with DSB-3, MitoTracker, and DAPI. White arrows point to PE and blue arrows to its co-localization with mitochondria marker, MitoTracker. The results are representative of three independent experiments. Scale bars represent 20 or 5 μm as indicated.

FIG. 6 demonstrates that SMAC is colocalized with PSD, interacts with and negatively regulates PSD activity. (A) Representative IF images for SMAC and PSD colocalization in A549 cells. Scale bars represent 10, 2.5 μm, as indicated. (B) PLA assay showing direct interaction between SMAC and PSD in A549 cells (scale bar represent 10 μm). Immunoblot (C) and quantitative analysis cells (D) of PSD and SMAC expression levels relative to WI-38 (n=3). PSD activity (n=6) (E) and PE levels (n=6) (F) were analyzed in the indicated cell lines. (G) Purified PSD and SMAC used in this study. (H) PSD interaction with SMAC analyzed using the MST method (n=4). Purified SMAC was fluorescently labelled with the NanoTemper BLUE protein-labelling kit. SMAC (1 μM) was incubated with purified PISD (78-625 nM) for 30 min at 37° C., then thermophoresis was measured as described in the Materials and Methods section hereinbelow. Kd=246_29 nM (n=3). (I) Inhibition of PSD activity by SMAC, as measured using the DSB-3 method (n=4). Results are the mean±SEM. *P≤0.05; **P≤0.01; ***P≤0.001; NS, nonsignificant.

FIG. 7 shows PSD- and SMAC-interacting partners. (A) Schematic presentation of peptide array and detection of their interaction with SMAC. (B) Glass-bound peptide array consisting of overlapping peptides derived from 15 SMAC-interacting proteins were incubated overnight with purified SMAC (0.3 μM) and then blotted with anti-SMAC antibodies (1:1000), followed by incubation with HRP-conjugated anti-mouse IgG and detection using a chemiluminescence kit. Dark spots represent binding of SMAC to peptides derived from SMAC-interacting proteins. (C) SMAC (0.3 μM) was incubated with purified PSD (0.4 μM) and blotted as in (B). The peptide spots where PSD prevented interaction with SMAC are circled. (D) Schematic presentation of peptide array and detection of their interaction with PSD. (E) Peptide array was blotted with purified PSD (0.15 μM) and then with anti-PSD antibodies as in (B). (F) PSD (0.15 μM) was preincubated with its interacting peptide representing spot 2F3 (20 μM), followed by array blotting with anti-PSD antibodies as in (E). The peptide spots where 2F3 peptide prevented interaction with PSD are circled. Each presented peptide array represents 2-3 similar experiments.

FIG. 8 shows the sequences of peptides that directly interacted with PSD (A) or that PSD prevented their interaction with SMAC (B). The interacting peptides are designated by their spot number and their protein of origin is also presented.

FIG. 9 shows that peptides identified from direct interact with PSD or PSD prevented their interaction with SMAC, labeled in the proteins they derived from A. Positions of peptides in space-filling models of ARNT (PDB_ID 4ZP4), peptide 2J14 (dark gray spheres), BIRC2, (PDB_ID 3T6P) peptide 1R14 (darker gray and TRAF2 (PDB_ID 1Ca4) 1K13 (dark gray) and 1K18 (dark gray). All were prepared with UCSF Chimera. B. Glass-bound peptide array consisting of overlapping peptides derived from 15 SMAC/PSD-interacting proteins was incubated overnight with PSD (0.15 μM) preincubated with its interacting peptide representing spot 1C11, followed by array blotting with anti-PSD antibodies. The peptide spots in the array that 1C11 peptide prevented or highly decreased their interaction with PSD are circled. This blotting was done in parallel with the control (PSD alone) shown in FIG. 7E.

FIG. 10 demonstrates that PSD-interacting peptides bind to PSD, inhibiting its activity, and when cell-penetrating and mitochondria- or nuclear-targeted, inhibit cell proliferation. (A) Fluorescently-labelled purified PSD (0.25 μM) was incubated with 2F3 (●) or 1C11 (◯) peptide (1-10 μM) for 30 min at 37° C.; then, MST was used and revealed Kd of 3.0 μM for both peptides (n=3). (B) Inhibition of PSD activity by the 2F3 (●) or 1C11 (◯) peptide. PSD activity was measured using the DSB-3 method. (C-E) Cell proliferation inhibition following incubation of A549 cells with the indicated mitochondria- or nuclear-targeted peptides for 24 h in a serum-free medium, and cell proliferation was assayed using the SRB method. Results are the means±SEM (n=3). (F) A549 cells were incubated for 90 min with 5 μM of the mitochondria- or nuclear targeted FITC-labelled peptides, immunostained with anti-SMAC antibodies and with DAPI and visualized by confocal microscope for subcellular localization. Arrows indicates nuclear (n=3). Scale bars represent 20

FIG. 11 demonstrates that PSD-interacting peptides do not inhibit cell growth of epithelial HaCaT cells (A-C) Cell growth inhibition following incubation of HaCaT cells with the indicated mitochondria- (black bars) or nuclear-targeted peptides (grey bars) for 24 h in a serum-free medium, and cell proliferation was assayed using the SRB method. Results are the means±SEM (n=3).

FIG. 12 shows cell proliferation inhibition by modified PSD interacting, mitochondria-targeted peptides. (A) A549 cells were incubated with the indicated concentration of 1C11-HTRA2-derived peptide targeted to the mitochondrion with the targeting sequence added to the N-terminus (N-Ter) or the C-terminus (C-Ter). (B) Mitochondrion targeted 1C11-HTRA2-derived peptide was modified by replacing the three amino acids in the C or the N-terminus of the peptide, or all amino acids in the peptide with the D-confirmation of the amino acid (underlined amino acids)). Following 24 h incubation in a serum-free medium, cell proliferation was assayed using the SRB method. (C) The concentration required for 50% inhibition of cell proliferation (IC50) is presented. Results are the means±SEM (n=3).

FIG. 13 shows tumor growth inhibition by PSD-interacting peptides targeted to the mitochondria in lung cancer xenograft. A549 cells were S.C. inoculated into female nude mice (7×10⁶ cells/mouse). Tumors sizes were measured (using a digital caliper) and volumes were calculated. When tumors volumes reached 60-100 mm3 (day 12), the mice were divided into 2 groups and xenografts were untreated (●) or treated with HtrA2/Omi-derived D-amino acid peptide targeted to the mitochondria (D-HTRA2-M, o). The tumors were injected 3 times a week to a final concentration of 50 μM. (B, C). Tumors of A549 cell xenografts were dissected, photographed (B) and weighted (C). (D-F) similar experiments as in A-C, except that the peptide was administrated by intravenous injection 2 times a week to a final concentration of 20 mg/ml. Results represent the means±SEM (n=8), **P≤0.01, ***P≤0.001.

FIG. 14 shows that peptide treatment reduced tumor cell proliferation Ki-67 expression and expression of SMAC and NF-kB. Sections of paraffin-embedded control and peptide treated tumors were IF stained using specific antibodies and subjected to quantification. Relative staining intensity for the nuclear proliferation marker, Ki-67 (and its (A), SMAC (B) and NF-kB (C). Results represent the means±SEM (n=3) ***P≤0.001. (G) Sections from the indicated tumors were stained with H & E, with some areas were magnified.

FIG. 15 shows PSD and SMAC expression in tumors and presence in the nucleus. Representative IHC staining of PSD (A, C) and SMAC (B, D) in sections derived from the indicated healthy (n=10) and cancer (n=20) tissues in tissue microarray slides (US Biomax). The percentages of patient samples stained at the intensities presented in the scale are at the top of the figure, and the quantitative analysis is also shown (C, D). Image enlargements are given to show protein nuclear localization (Aa, Bb). Data shown are representative of three independent experiments. Results represent the means±SEM, *P<0.05, **P<0.01, ***P<0.001. Scale bars represent 50 or 10 μm as indicated.

FIG. 16 demonstrates that PSD is present in cell nucleus. (A) For PSD and SMAC sub-cellular localization in cells, nuclear extracts were prepared from NCI-H-1563 cells using a nuclear/cytosol fractionation kit (Biovision, Milpitas, CA), following the manufacturer's instructions. Before centrifugation (Total) and after centrifugation (16,000 g, 10 min), the supernatant (cytosolic fraction), and pellet (nuclear fraction) were re-suspended in the original volume and subjected to immunoblotting for SMAC, PSD, GAPDH (cytosolic), ATPsyn5a (mitochondria) and histone 4 (H4, nuclear fraction) (A) The total, nuclear-free and nuclear fractions were subjected to immunoblotting and quantitative analysis of the PSD levels (black bars) and to PSD activity assay (white bars) as described in the Materials and Methods section. Results represent the means±SEM (n=3) (B).

FIG. 17 is a proposed mode of PSD activity regulation of and by SMAC, affecting cellular PE levels and PE multifunction. A cell with ER, mitochondria and nucleus presenting SMAC, PSD and PE activities. Phospholipid biosynthesis: Mitochondria with proposed SMAC regulation of PSD activity, thereby regulating PE synthesis at ER-mitochondria contact site (MAM). PS is produced in the ER from PC and PE, and PS then is transferred to the mitochondria, where it is converted by PSD into PE, and is depleted in other cell compartments including membranes and the nucleus. SMAC regulation of mitochondria-mediated apoptosis and possible inhibition of SMAC release by PSD are proposed. PSD, SMAC and their identified interacting proteins in the nucleus, controlling PE production in nucleus. The PE multi-functions in the mitochondria, cell membrane, and nucleus are presented.

### DETAILED DESCRIPTION OF THE INVENTION

The present invention provides compositions and methods for treating various types of cancer diseases via modulation of phospholipid metabolism in cancerous cells, particularly via modulating the expression and/or activity of the enzyme phosphatidylserine decarboxylase (PSD).

### Definitions

The terms “phosphatidylserine decarboxylase” and its abbreviation “PSD” are used herein interchangeably and refer to an enzyme that catalyzes the synthesis of phosphatidylethanolamine (PE) from phosphatidylserine (PS), and plays a central role in phospholipid metabolism and in the inter-organelle trafficking of phosphatidylserine. According to certain embodiments, the term refers to the mammalian enzyme, particularly to the mitochondrial human enzyme EC:4.1.1.65 (B4DPS3_HUMAN). According to certain embodiments, the PSD enzyme comprises the amino acid sequence set forth in SEQ ID NO:32.

As used herein, the term “modulating PSD activity”, refers to modulating, according to certain embodiments inhibiting, the enzymatic activity of PSD of converting PS to PE as well as to indirect modulation of its activity by interfering with its binding to other proteins, thereby modulating the activity of the PSD-associated protein complex (“signaling activity”). The peptides of the invention may further modulate the PSD location within the cell.

As used herein, the terms “HTRA2”, “HtrA2” and “HtrA2/Omi” are used herein interchangeably and refer to Serine peptidase 2. Serine protease promotes cell death either by direct binding to IAP proteins leading to increased caspase activity or a caspase-independent and serine protease activity-dependent mechanism. It can be found in the mitochondrion intermembrane space, and the nucleus (Vande W L, et al., 2008. Cell Death Differ 15, 453-460).

As used herein, the term “BIRC2” refers to Baculoviral IAP2 repeat. This protein acts as an E3 ubiquitin-protein ligase. It is a regulator of NF-kappa-B signaling, apoptosis, cell proliferation, cell invasion, and metastasis. It modulates inflammatory signaling, and is present in the cytosol, nucleus (Samuel T, et al., 2005. Cancer Res 65, 210-218) and plasma membrane.

As used herein, the term “TRAF-2” refers to TNF receptor associated factor 2. It regulates activation of NF-κB and JNK, cell survival, and apoptosis. Constituent of several E3 ubiquitin-protein ligase complexes regulates BIRC2, BIRC3, RIPK1, and TICAM1 protein levels by inhibiting their autoubiquitination. It is present in the Cytosol.

As used herein, the term “MTFR1” refers to mitochondrial fission regulator 1. It promotes mitochondrial fission, and deficiency of Mtfr1 results in oxidative DNA damage. It is present in the mitochondria.

As used herein, the term “MAML2” refers to mastermind like transcriptional coactivator 2 truncated poly Q. It is a transcriptional coactivator for NOTCH proteins. It promotes proliferative signaling during neurogenesis. It is present in the nucleus (Wu L, et al., 2002. Mol Cell Biol 22, 7688-7700).

As used herein, the term “ARNT” refers to aryl hydrocarbon receptor nuclear translocator isoform 1 (known as HIF-10); ARNT is required for the ligand-binding subunit to translocate from the cytosol to the nucleus after ligand binding. When bound, the ligand translocated from the cytosol to the nucleus involve its translocator (AREN). Identified as the beta subunit of a heterodimeric transcription factor, hypoxia-inducible factor 1 (HIF1), and functions as a transcriptional regulator of the adaptive response to hypoxia. It is present in the nucleus (Seok S H, et al. 2017. Proc Natl Acad Sci USA 114, 5431-5436).

As used herein, the term “NR4A1” refers to nuclear receptor subfamily 4 group A. It is a member of the steroid-thyroid hormone-retinoid receptor superfamily. It acts as a nuclear transcription factor. Translocation of the protein from the nucleus to mitochondria induces apoptosis. It is present in the nucleus and the mitochondrion (Zhang L, et al., 2018. J Diabetes Res 2018, 9363461).

As used herein, the term “treatment” or “treating” refers to clinical intervention designed to alter the natural course of the individual or cell being treated during the course of clinical pathology, e.g., cancer. Desirable effects of treatment include decreasing the rate of disease progression (delaying progression of a disease), ameliorating or palliating the disease state, and remission or improved prognosis of the disease. For example, an individual is successfully “treated” if one or more symptoms associated with cancer are mitigated or eliminated, including, but are not limited to, reducing the proliferation of cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

The terms “cell growth” and “cell proliferation” are used herein interchangeably and refer to the number of viable cells of a particular type observed after a certain growth period.

The terms “inhibit”, “decrease”, “reduce” and ‘silence” with regard to the expression or activity of PSD are used herein interchangeably and includes any decrease in expression or protein activity or level of the PSD gene or mRNA or protein encoded by the PSD. According to certain embodiments, inhibition of PSD activity refers to modulation or inhibition of PSD activity within a mammalian cell, particularly within the nucleus and/or mitochondria, resulting in an inhibition/decrease/reduction of cell growth (proliferation). The decrease may be of at least 20%, 30%, 40%, 50%, 60%, 70%, 80%, 90%, 95% or 99% or more, particularly in cancerous cells, as compared to the expression and/or activity of PSD in non-cancerous cells wherein PSD has not been modulated.

As used herein, the term “peptide” indicates a sequence of amino acids linked by peptide bonds. Peptides according to some embodiments of the present invention consist of 10-50 amino acids, for example 15-35 amino acids or 20-25 amino acids.

In some embodiments, a peptide according to the present invention is up to 30 amino acids, for example up to 29 amino acids, 28 amino acids, 27 amino acids, 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, or up to 10 amino acids. Each possibility represents a separate embodiment of the invention.

The term “amino acid” refers to compounds, which have an amino group and a carboxylic acid group, preferably in a 1,2- 1,3-, or 1,4-substitution pattern on a carbon backbone. α-Amino acids are most preferred, and include the 20 natural amino acids (which are L-amino acids except for glycine) which are found in proteins, the corresponding D-amino acids configuration, the corresponding N-methyl amino acids, side chain modified amino acids, the biosynthetically available amino acids which are not found in proteins (e.g., 4-hydroxy-proline, 5-hydroxy-lysine, citrulline, ornithine (Orn), canavanine, djenkolic acid, β-cyanoalanine), and synthetically derived α-amino acids, such as aminoisobutyric acid, norleucine (Nle), norvaline (NorVal, Nva), homocysteine and homoserine. β-Alanine and γ-amino butyric acid are examples of 1,3 and 1,4-amino acids, respectively, and many others as well known to the art.

Some of the amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by one-letter codes or three-letter codes according to IUPAC conventions. When there is no indication, the L isomer was used. The D isomers are indicated by “D” or “(D)” before the residue abbreviation. The amino acids of the peptides of the present invention can be all L-amino acids, all D-amino acids, or comprise a combination of L- and D-amino acids. Each possibility represents separate embodiment of the present invention.

As used herein, an “amino acid residue” means the moiety which remains after the amino acid has been conjugated to additional amino acid(s) to form a peptide, or to a moiety (such as a cell penetrating moiety and/or mitochondria and/or nuclear targeting moiety), typically through the alpha-amino and carboxyl of the amino acid.

As used herein, the terms “targeting moiety” and “localization moiety” with reference to targeting of a peptide of the invention to the nucleus and/or mitochondria are used herein interchangeably and refer to a molecule which is able to target the peptide to the specific organelle and facilitate or enhance its penetration into the nucleus or mitochondria. The targeting moiety typically enhances the permeability of the peptide, i.e., its ability to penetrate, pervade, or diffuse through a barrier or membrane, typically a phospholipid membrane. The nuclear and/or mitochondrial targeting moiety may also enhance the penetration of the peptide through the plasma membrane. Additionally, or alternatively, a cell penetrating moiety specifically designed to enhance the permeability of the peptide through the plasma membrane is added to the targeting moiety.

In the course of the research leading to the present invention, it has been discovered that inhibiting the activity of PSD in cancer cells and tumors comprising same results in inhibited growth of the cancer cells. This finding is highly unexpected in view of previous finding of the inventor of the present invention and co-workers, according to which increased activity of PSD (resulting from depletion of SMAC/Diablo expression) was followed by inhibition of cancer cell growth and proliferation.

The present invention thus discloses novel peptides capable of binding to and modulating the activity of PSD, pharmaceutical composition comprising same and use thereof for treating cancer.

According to certain aspects, the present invention provides an agent capable of modulating the activity of a phosphatidylserine decarboxylase (PSD).

According to certain exemplary embodiments, the PSD is mitochondrial PSD.

According to further aspects, the present invention provides a method for treating cancer in a subject in need thereof, comprising administering to the subject a therapeutically effective amount of at least one agent capable of modulating the activity of phosphatidylserine decarboxylase (PSD) or a pharmaceutical composition comprising same.

According to yet further aspect, the preset invention provides at least one agent capable of modulating the activity of phosphatidylserine decarboxylase (PSD) or a pharmaceutical composition comprising same for use in treating cancer in a subject in need thereof.

According to certain embodiments, modulating the activity of PSD comprises inhibiting its activity of converting PS to PE. According to certain additional or alternative embodiments, modulating the activity of PSD comprises inhibiting its interaction with other proteins.

According to certain embodiments, the agent is capable of modulating the activity of PSD within a mammalian cell.

According to certain embodiments, the agent capable of modulating the activity of PSD is selected from the group consisting of a peptide, a small molecule, a nucleic acid molecule and any combination thereof.

According to certain exemplary embodiments, the agent is a peptide capable of interacting with PSD. According to some embodiments, the peptide is a synthetic or recombinant peptide.

According to some embodiments, the peptide modulating the activity of PSD is derived from a polypeptide or protein interacting with PSD.

According to certain exemplary embodiments, the protein interacting with PSD is selected from the group consisting of MAML2, HTRA2, BIRC2, TRAF-2, MTFR1, and ARNT. Each possibility represents a separate embodiment of the present invention.

According to certain embodiments, the peptide modulating the activity of PSD comprises an amino acid sequence having at least 80% identity over at least 97% coverage to an amino acid sequence selected from the group consisting of SEQ ID NOs:1, 4, 5, 2, 6-9, and 11-14, an analog, derivative or a fragment thereof. Each possibility represents a separate embodiment of the present invention.

According to certain embodiments, the peptide comprises an amino acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more homologous, or identical, over at least about 97%, at least about 97.5%, at least about 98%, at least about 98.5%, at least about 99%, at least about 95.5% or 100% coverage to an amino acid sequence selected from the group consisting of SEQ ID NOs:1, 4, 5, 2, 6-9, and 11-14, an analog, derivative or a fragment thereof. Each possibility represents a separate embodiment of the present invention.

According to certain embodiments, the peptide modulating the activity of PSD comprises an amino acid sequence having at least 80% identity over at least 23 contiguous amino acids of a sequence selected from the group consisting of SEQ ID NOs:1, 4, 5, 2, 6-9, and 11-14, an analog, derivative or a fragment thereof. Each possibility represents a separate embodiment of the present invention.

According to certain embodiments, the peptide comprises an amino acid sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more homologous, or identical over at least 23, at least 24, or at least 25 contiguous amino acids of a sequence selected from the group consisting of SEQ ID NOs:1, 4, 5, 2, 6-9, and 11-14, an analog, derivative or a fragment thereof.

According to certain embodiments, the peptide modulating the activity of PSD comprises an amino acid sequence selected from the group consisting of SEQ ID NOs:1, 4, 5, 2, 6-9, and 11-14, an analog, derivative or a fragment thereof. According to certain exemplary embodiments, the peptide modulating the activity of PSD consists of the amino acid sequence set forth in any one of SEQ ID NOs:1, 4, 5, 2, 6-9, and 11-14. Each possibility represents a separate embodiment of the present invention.

According to certain embodiments, the peptide modulating the activity of PSD, designated herein IC11 is derived from the protein HTRA2. According to certain embodiments, peptide IC11 comprises an amino acid sequence having at least 80% identity over at least 97% coverage to the amino acid sequence set forth in SEQ ID NO:1.

According to some embodiments, the peptide modulating the activity of PSD comprises the amino acid sequence set forth in SEQ ID NO:1. According to some embodiments, the peptide modulating the activity of PSD consists of the amino acid sequence set forth in SEQ ID NO:1.

According to certain embodiments, the peptide modulating the activity of PSD, designated herein 2F3, is derived from the protein MAMAL2. According to certain embodiments, peptide 2F3 comprises an amino acid sequence having at least 80% identity over at least 97% coverage to the amino acid sequence set forth in SEQ ID NO:4.

According to some embodiments, the peptide modulating the activity of PSD comprises the amino acid sequence set forth in SEQ ID NO:4. According to some embodiments, the peptide modulating the activity of PSD consists of the amino acid sequence set forth in SEQ ID NO:4.

According to certain embodiments, the peptide modulating the activity of PSD, designated herein 218, is derived from the protein MAMAL2. According to certain embodiments, peptide 218 comprises an amino acid sequence having at least 80% identity over at least 97% coverage to the amino acid sequence set forth in SEQ ID NO:7. According to some embodiments, the peptide modulating the activity of PSD comprises the amino acid sequence set forth in SEQ ID NO:7. According to some embodiments, the peptide modulating the activity of PSD consists of the amino acid sequence set forth in SEQ ID NO:7.

According to certain embodiments, the peptide modulating the activity of PSD is targeted to the nucleus and/or to the mitochondrion of a cell. According to certain embodiments, the cell is within a cell culture. According to certain additional or alternative embodiments, the cell is within a subject. According to certain exemplary embodiments the cell is a mammalian cell, particularly human cell. According to further certain exemplary embodiments, the mammalian cell is a cancerous cell.

According to certain embodiments, the peptide modulating the activity of PSD further comprises at least one nuclear and/or mitochondrial targeting moiety.

According to certain exemplary embodiments, the peptide modulating the activity of PSD comprises an amino acid sequence having at least 80% identity over at least 97% coverage to an amino acid sequence selected from the group consisting of SEQ ID NOs:1, 4, 5, 2, 6-9, and 11-14, an analog, derivative or a fragment thereof linked to a mitochondrial and/or nuclear targeting moiety.

The mitochondrial and/or nuclear targeting moiety can be peptidic or non-peptidic, and is covalently linked to the peptide modulating PSD activity directly or via linker. The targeting moiety may be linked to the inhibitory peptide at any position, as long as the resulting peptide preserves the capability to modulate PSD activity. According to certain exemplary embodiments, the targeting moiety is linked to the N- or the C-terminus of the peptide modulating the activity of PSD. When the peptide comprises a combination of mitochondrial and nuclear targeting moieties, each moiety may be independently linked to the N- or C-terminus of the peptide or the mitochondrial and nuclear targeting moieties can be linked in tandem to the N- or C-terminus of the peptide. According to certain exemplary embodiments, each of the nuclear and mitochondrial targeting moiety also enhances the permeability of the inhibitory peptide through the cell membrane.

The peptide modulating PSD activity can be synthetic or recombinant.

According to certain embodiments, the peptide modulating the activity of PSD comprises an amino acid sequence having at least 80% identity over at least 97% coverage to an amino acid sequence selected from the group consisting of SEQ ID NOs:1, 4, 5, 2, 6-9, and 11-14, an analog, derivative or a fragment thereof linked to a mitochondrial targeting moiety.

According to certain embodiments, the peptide modulating the activity of PSD comprises an amino acid sequence having at least 80% identity over at least 97% coverage to an amino acid sequence selected from the group consisting of SEQ ID NOs:1, 4, 5, 2, 6-9, and 11-14, an analog, derivative or a fragment thereof linked to a nuclear targeting moiety.

Any mitochondrial and/or nuclear targeting moiety can be used according to the teachings of the present invention. According to certain exemplary embodiments, the mitochondrial targeting moiety comprises the amino acid sequence D-Arg-Dmt-Orn-Phe-NH₂, where Dmt is 2,6-dimethyl-L-tyrosine (SEQ ID NO:15). According to certain exemplary embodiments, the nuclear targeting moiety is the tetrapeptide RrRK, wherein r is D-arginine (SEQ ID NO:16).

According to some embodiments, the peptide modulating PSD activity further comprises a cell penetration moiety enhancing the permeability of the peptide through the cell plasma membrane. Any cell penetrating moiety as is known in the art can be used according to the teachings of the present invention. According to certain exemplary embodiments, the cell penetration moiety is a peptide (CPP).

According to certain exemplary embodiments, the present invention provides a synthetic peptide comprising (i) a peptide modulating the activity of PSD comprising an amino acid sequence having at least 80% identity over at least 97% coverage to the amino acid sequence set forth in SEQ ID NO:1 and (ii) a mitochondrial targeting moiety comprising the amino acids sequence set forth in SEQ ID NO:15 or a nuclear targeting moiety comprising the amino acids sequence set forth in SEQ ID NO:16 directly connected to the C-terminus of the peptide modulating the activity of PSD. According to certain further exemplary embodiments, the peptide modulating the activity of PSD comprises SEQ ID NO:1. According to some embodiments, the peptide modulating PSD activity consists of SEQ ID NO:1.

According to certain exemplary embodiments, the peptide comprises an amino acid sequence set forth in any one of SEQ ID NOs:25 and 28.

According to certain exemplary embodiments, the present invention provides a synthetic peptide comprising (i) a peptide modulating the activity of PSD comprising an amino acid sequence having at least 80% identity over at least 97% coverage to the amino acid sequence set forth in SEQ ID NO:4 and (ii) a mitochondrial targeting moiety comprising the amino acids sequence set forth in SEQ ID NO:15 directly connected to the C-terminus of the peptide modulating the activity of PSD. According to certain further exemplary embodiments, the peptide modulating the activity of PSD comprises SEQ ID NO:4. According to some embodiments, the peptide modulating PSD activity consists of SEQ ID NO:4.

According to certain exemplary embodiments, the peptide comprises an amino acid sequence selected from the group consisting of SEQ ID NOs:17, SEQ ID NO:19 and SEQ ID NO:20. According to certain further exemplary embodiments, the present invention provides a synthetic peptide comprising (i) a peptide modulating the activity of PSD comprising an amino acid sequence having at least 80% identity over at least 97% coverage to the amino acid sequence set forth in SEQ ID NO:4 and (ii) a nuclear targeting moiety comprising the amino acids sequence set forth in SEQ ID NO:16 independently directly connected to the C- or N-terminus of the peptide modulating the activity of PSD. According to certain further exemplary embodiments, the peptide modulating the activity of PSD comprises SEQ ID NO:4. According to some embodiments, the peptide modulating PSD activity consists of SEQ ID NO:4.

According to additional certain exemplary embodiments, the present invention provides a synthetic peptide comprising (i) a peptide modulating the activity of PSD comprising an amino acid sequence having at least 80% identity over at least 97% coverage to the amino acid sequence set forth in SEQ ID NO:7 and (ii) a mitochondrial targeting moiety comprising the amino acids sequence set forth in SEQ ID NO:15 or a nuclear targeting moiety comprising the amino acids sequence set forth in SEQ ID NO:16 directly connected to the C-terminus of the peptide modulating the activity of PSD. According to certain further exemplary embodiments, the peptide modulating the activity of PSD comprises SEQ ID NO:7. According to some embodiments, the peptide modulating PSD activity consists of SEQ ID NO:7.

According to certain exemplary embodiments, the peptide comprises an amino acid sequence set forth in any one of SEQ ID NOs:21 and 24.

According to certain embodiments, the peptide modulating the activity of PSD (with or without mitochondrial and/or nuclear targeting peptide) further comprises at least one additional moiety selected from the group consisting of cell penetration moiety, a detectable label and a carrier. Each possibility represents a separate embodiment of the present invention. In some embodiments, the cell penetration moiety is a fatty acid residue. The additional moiety can be linked to the inhibitor peptide directly or via a linker. Each possibility represents a separate embodiment of the present invention.

According to certain embodiments, the C-terminus of the peptide modulating PSD activity with or without a mitochondrial and/or nuclear targeting peptide is a modified carboxy terminal group selected from the group consisting of an amide, ester and alcohol group. Each possibility represents a separate embodiment of the present invention.

In some embodiments, the N-terminus of the peptide modulating PSD activity with or without a mitochondrial and/or nuclear targeting peptide is modified with an amino terminal blocking group. In some embodiments, the amino terminal blocking group is selected from the group consisting of an acetyl and alkyl. Each possibility represents a separate embodiment of the present invention.

In some embodiments, the amino- or carboxy-terminus of the inhibitory peptides disclosed herein is modified by the addition of a moiety enhancing the cell permeability (cell penetrating moiety).

Non-limitative examples of cell permeability moieties include hydrophobic moieties such as lipids, fatty acids, steroids and bulky aromatic or aliphatic compounds. According to certain embodiments, the cell penetrating moiety is a peptide (CPP).

In some embodiments, the cell permeability moiety is covalently linked to the N- or C-terminus of the peptide via a direct bond. In other embodiments, the cell permeability moiety is covalently linked to the N- or C-terminus of the peptide via a linker. In some embodiments, the cell permeability moiety is a fatty acid residue. In some embodiments, the fatty acid residue is selected from C12-C20 fatty acids. In some particular embodiments, the fatty acid residue is a myristoyl group (Myr). In additional particular embodiments, the fatty acid residue is a stearoyl group (Stear). In yet additional embodiments, the fatty acid residue is a palmitoyl group (Palm). In further embodiments, the cell permeability peptide comprises transferrin-receptor binding domain (Tf) comprising the amino acid sequence set forth in SEQ ID NO:36 (HAIYPRH) or a fragment thereof. In some meboidments, the cell permeability peptide comprises the Drosophila antennapedia (Antp) domain comprising the amino acid sequence set forth in SEQ ID NO:37 (RQIKIWFQNRRMKWKK) or a fragment thereof. The procedures utilized to construct peptide compounds of the present invention generally rely on the known principles and methods of peptide synthesis, such as solid phase peptide synthesis, partial solid phase synthesis, fragment condensation and classical solution synthesis.

Some of the peptides of the present invention, that do not comprise non-coded amino acids, can be synthesized using recombinant methods know in the art. Peptides comprising targeting and/or penetrating moieties may be synthesized chemically or alternatively may be produced recombinantly and coupled synthetically with the desired moiety.

The peptides of the invention can be used in the form of pharmaceutically acceptable salts. As used herein the term “salts” refers to both salts of carboxyl groups and to acid addition salts of amino or guanido groups of the peptide molecule. The term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans. Pharmaceutically acceptable salts include those salts formed with free amino groups such as salts derived from non-toxic inorganic or organic acids such as acetic acid, citric acid or oxalic acid and the like, and those salts formed with free carboxyl groups such as salts derived from non-toxic inorganic or organic bases such as sodium, calcium, potassium, ammonium, calcium, ferric or zinc, isopropylamine, triethylamine, procaine, and the like.

Analogs and derivatives of the peptides are also within the scope of the present application.

“Derivatives” of the peptides of the invention as used herein cover derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it, and do not adversely affect the immunogenic properties thereof.

These derivatives may include, for example, aliphatic esters of the carboxyl groups, amides of the carboxyl groups produced by reaction with ammonia or with primary or secondary amines, N-acyl derivatives of free amino groups of the amino acid residues, e.g., N-acetyl, formed by reaction with acyl moieties (e.g., alkanoyl or carbocyclic aroyl groups), or O-acyl derivatives of free hydroxyl group (e.g., that of seryl or threonyl residues) formed by reaction with acyl moieties.

“Analogs” of the peptides of the invention as used herein cover compounds which have the amino acid sequence according to the invention except for one or more amino acid changes, typically, conservative amino acid substitutions.

In some embodiments, an analog has at least about 75% identity to the sequence of the peptide of the invention, for example at least about 80%, at least about 85%, at least about 90%, at least about 95%, or at least about 99% identity to the sequence of the peptide of the invention.

Conservative substitutions of amino acids as known to those skilled in the art are within the scope of the present invention. Conservative amino acid substitutions include replacement of one amino acid with another having the same type of functional group or side chain e.g., aliphatic, aromatic, positively charged, negatively charged.

Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K), Histidine (H);
5) Isoleucine (I), Leucine (L), Methionine (M), Valine (V); and
6) Phenylalanine (F), Tyrosine (Y), Tryptophan (W).

Analogs according to the present invention may comprise also peptidomimetics. “Peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with another covalent bond. A peptidomimetic according to the present invention may optionally comprise at least one bond which is an amide replacement bond such as urea bond, carbamate bond, sulfonamide bond, hydrazine bond, or any other covalent bond. The design of appropriate analogs may be computer assisted. Analogs are included in the invention as long as they remain pharmaceutically acceptable and their activity is not damaged.

The inhibitory agents of the present invention, particularly the inhibitory peptides described hereinabove can be administered to a subject per se, or in a pharmaceutical composition where they are mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent accounting for modulating the activity of the enzyme PSD, particularly within a nucleus or a mitochondrion of a cancerous cell. According to certain exemplary embodiments, the active ingredient of the invention is a peptide as described herein, inhibiting the proliferation of cancerous cell and/or reducing the growth of a cancerous tumor.

The phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, or intranasal injections.

Alternately, one may administer the pharmaceutical composition in a local rather than systemic manner, for example, by intra-articular injections or by microinjections, under arthroscopy, into the inflammatory synovial tissue (i.e., in situ).

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid agent) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975. In: “The Pharmacological Basis of Therapeutics”, Ch. 1 p. 1).

Dosage amount and interval may be adjusted individually to provide plasma levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as the U.S. Food and Drug Administration (FDA) approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

The inventor of the present invention and co-workers have previously demonstrated that SMAC/Diablo depletion in cancer cells using specific si-RNA led to multiple effects, including reduced cell proliferation and tumor growth, decreased phospholipid levels, and induced cell differentiation.

During the course of the research leading to the present invention, it was shown that depletion of SMAC/Diablo using CRISPR/Cas results in inhibition of cancer cell proliferation, but not in inhibition of cells considered as immortalized but noncancerous; and in an increase in mitochondrial PE and a decrease in the level of other phospholipids (PLs). It was shown that SMAC regulates the levels of PE via direct interaction with mitochondrial PSD inhibiting its activity, and PSD-interacting sites with SMAC and three nuclear proteins were identified. The interacting sequences were revealed, and the present invention now shows that these interacting sequences, inhibit PSD activity and cell proliferation.

Without wishing to be bound by any specific theory or mechanism of action, PSD “trapping” by its interacting peptide in the mitochondria or the nucleus, may result in decreased PS levels, known to contribute to cancer immunosuppression and inflammation. PS, a substrate of PSD, is exposed only on tumor cells but not healthy cells, and is important for cell survival, growth, proliferation and cancer-related symptoms (Chang W, et al., 2020. Theranostics 10(20), 9214-9229). Its exposure on the surface of tumor cells prevents immune reaction by ligation of PS to receptors present on dendritic cells, macrophages and T cells. PS receptors function as mediators to invoke immune suppression (Dayoub, A S, et al., 2020. Cell Commun Signal 18(1), 29).

Immunosuppression and inflammation contribute to the creation of an environment that facilitates tumor growth and proliferation. Both preclinical and early phase clinical trials using PS targeting agents, including monoclonal antibodies, antibody-drug conjugations, liposomal carriers and natural products, have shown potential antitumor activities. Thus, the disclosure of the presented invention of PSD-targeting peptides, inhibiting tumor growth, reducing inflammation and immunosuppression leads to their use as anti-cancer drugs, which may modulate PS levels and thereby eliminate the PS-induce immunosuppression in cancer cells.

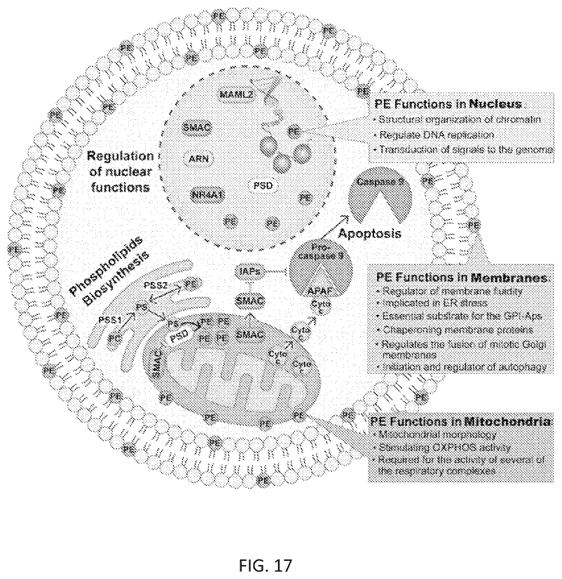

PE is not only a membrane component, but also is considered a signaling molecule that modulates structural organization of chromatin, nucleic acid synthesis and DNA replication (Alessenko A V, and Burlakova E B, 2002. Bioelectrochemistry 58, 13-21; Maraldi N M, et al., 1993. J Cell Sci 104 (Pt 3), 853-859. (FIG. 17). Regulating PE levels, via peptides modulating PSD activity as disclosed in the present invention as well as via SMAC, resulted in changes in phospholipid levels in the cell, ER and nucleus, with PE being depleted in these membranes, but accumulating in the mitochondria.

Finally, while approaches for cancer treatment targeting metabolism, signal transduction and mutated proteins have long been addressed, the present invention shows for the first time that affecting phospholipid synthesis, found essential for cancer growth and tumor progression, may be used as anticancer therapy.

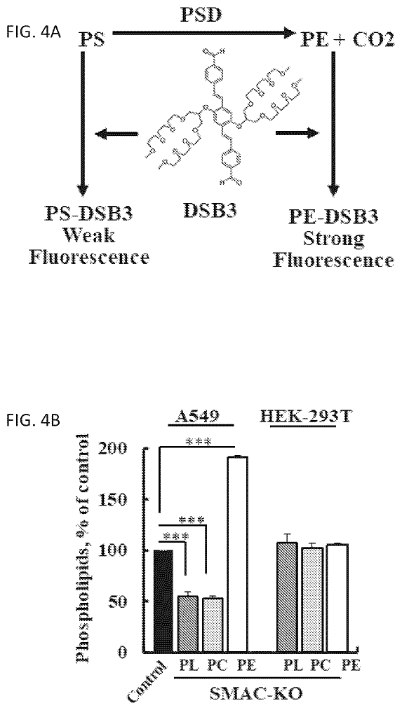

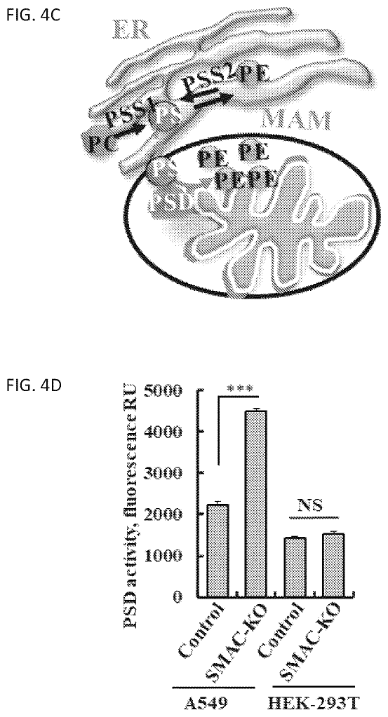

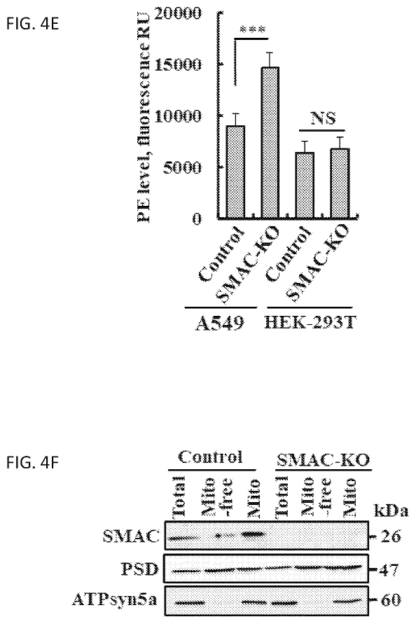

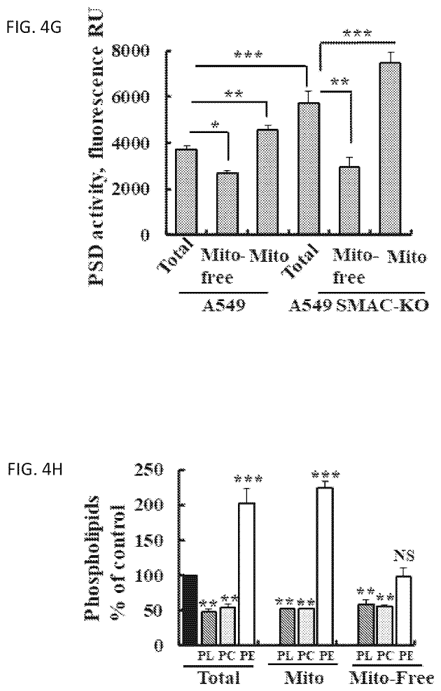

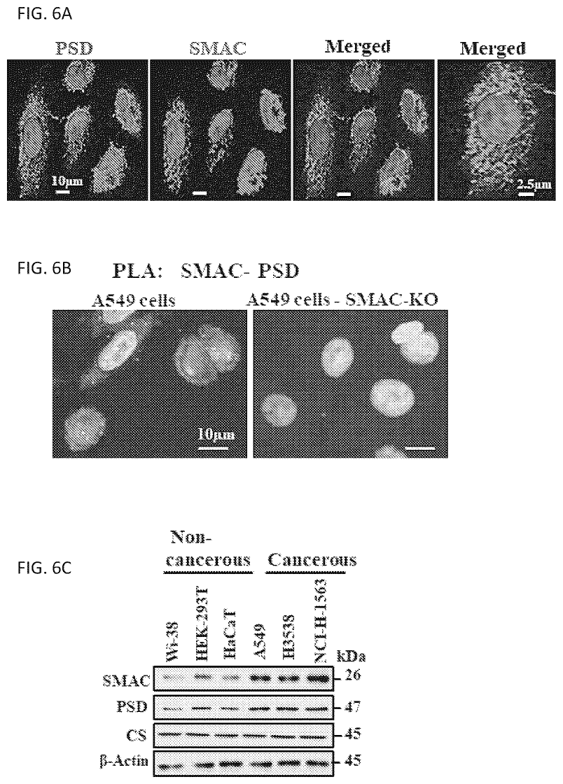

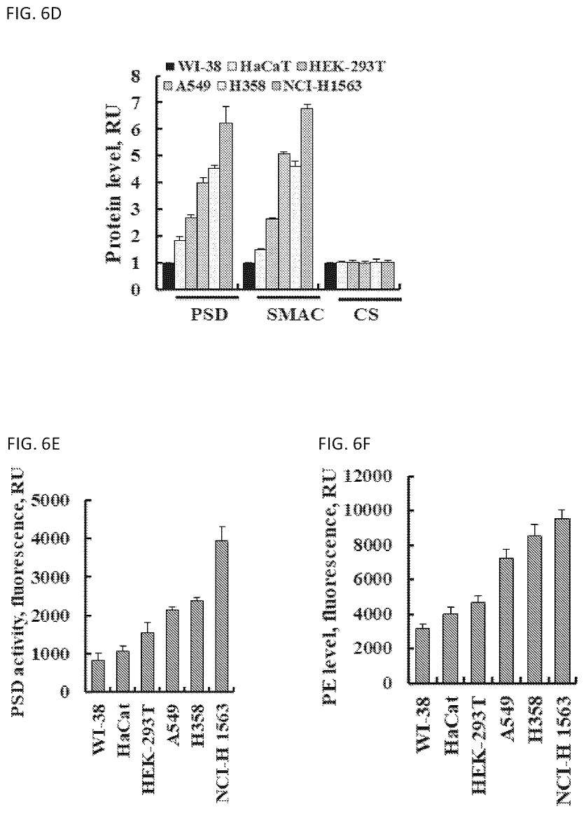

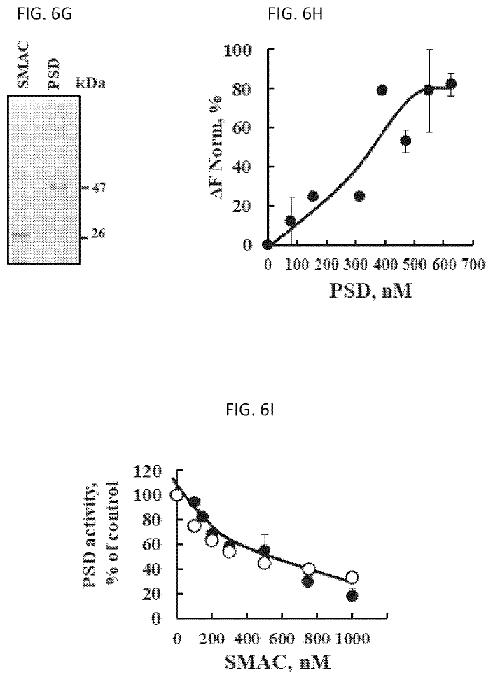

In mammalian cells, PE can be synthesized by two major pathways: the Kennedy pathway, where the final step takes place on ER membranes by choline/ethanolamine phosphotransferase 1 (CEPT1) (Vance J E, 1990. J Biol Chem 265, 7248-7256), and by decarboxylation of PS by the mitochondrial PSD (Percy A K, et al., 1983. Arch Biochem Biophys 223, 484-494). PS is produced in the ER from PC and PE by PSS1 and PSS2, respectively, and then transferred to the mitochondria, where it is converted into PE by PSD (FIG. 4C and FIG. 17). This key protein in PE synthesis is localized, as is SMAC in the IMS (FIG. 6A, B). SMAC directly interacts with and modulates PSD activity, as demonstrated by MST and PLA methods, and SMAC-inhibiting PSD activity. In agreement with the previous finding of the inventor (Paul et al., 2018, ibid; 2WO 019/021289), SMAC deletion both in tumors and in CRISPR/Cas9 SMAC-knock-out (KO) cancer cells, resulted in about twofold decrease in PLs and PC levels, while PE levels were increased twofold in the mitochondria. These, together with increased PSD activity in the absence of SMAC, suggests that in the absence of SMAC, PSD is activated.

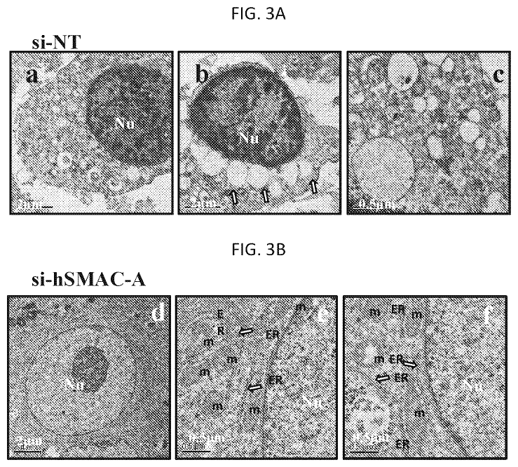

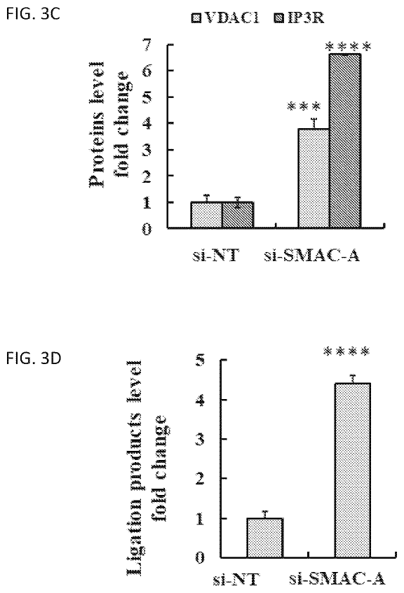

The increase in ER-mitochondria contact sites (MAM), the major site of PE synthesis, upon SMAC depletion (FIGS. 3 and 4C), suggests the formation of more sites for PS transport from the ER to the mitochondria and its conversion to PE by PSD. This, together with the high increase in mitochondria PE levels, suggests that in the absence of SMAC, the flux of PS from the ER to the mitochondria is increased. This, in turn, would result in depletion of PC, PE serving as PS precursors in the ER, as reflected in their decreased levels in the tumors and in SMAC-KO cells.

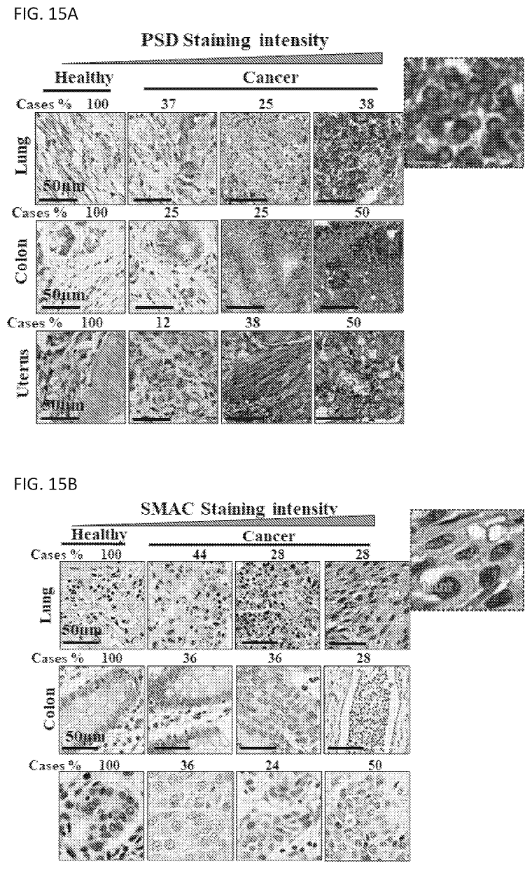

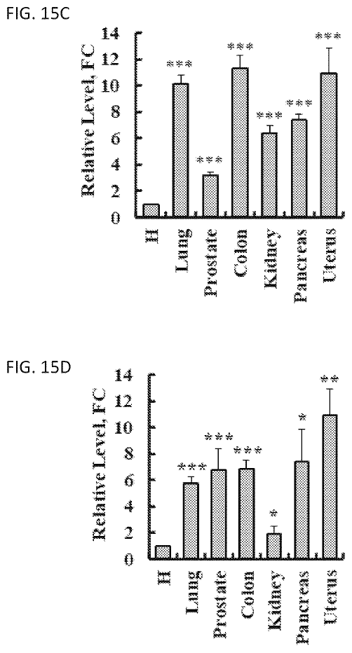

The differences between cancer and non-tumorigenic cells with respect to the differential effects of SMAC-KO in HEK293T and A549 can be related to the presence of higher levels of PE, PSD in tumors from different cancer types (FIG. 15), and SMAC in cancer cell (FIG. 6E-G, Paul et al., 2018, ibid). In this respect, it has been reported that PE levels are elevated in several cancers (Podo F. 1999. NMR Biomed 12, 413-439) and PE is asymmetrically distributed in the inner leaflet of the plasma membrane of noncancer mammalian cells, but not in cancer cells which have lost their capacity to maintain PE asymmetry (Vance and Tasseva 2013, ibid). Additionally, IMS serine protease LACTB, leading to PSD degradation, changes mitochondrial lipid metabolism in certain cancer cells, but not in non-tumorigenic differentiated cells (Keckesova Z, et al., 2017. Nature 543, 681-686). Furthermore, LACTB-mediated tumor suppression by increasing mitochondrial lipid metabolism (Cucchi D and Mauro C, 2017. Cell Death Differ 24, 1137-1139). Finally, acute myeloid leukemia (AML) cells lacking PSD failed to form tumors (Seneviratne A K, et al., 2019. Cell Stem Cell 24, 1007).

Another important finding is that PE not only a membrane component, but also a signaling molecule. PE is the only phospholipid synthesized in mitochondria in addition to the ER (Vance and Tasseva 2013, ibid). PE has remarkable functions and has roles in autophagy (Kabeya Y, et al., 2004. J Cell Sci 117, 2805-2812; Rockenfeller P, et al., 2015. Cell Death Differ 22, 499-508); ferroptosis; Parkinson's disease; and cancer (Patel D and Witt S N, 2017. Oxid Med Cell Longev 2017, 4829180). Overall, PE's numerous functions include, but are not limited to membranes, cell functions, and nuclear functions (FIG. 17).

### Membranes

PE is a regulator of membrane fluidity (Dawaliby R, et al., 2016. J Biol Chem 291, 3658-3667), with the ratio of PC to PE influencing membrane integrity and diseases like steatohepatitis (Li Z, et al., 2006. Cell Metab 3, 321-331). The IMM is enriched in PE compared to other membranes, and a decrease in the mitochondrial content of PE profoundly alters mitochondrial morphology in mammalian cells (Steenbergen R, et al., 2005. J Biol Chem 280, 40032-40040). PE is a lipid chaperone that assists in the folding of certain membrane proteins (Bogdanov M, et al., 1999. J Biol Chem 274, 12339-12345). Finally, PE is an essential substrate for the synthesis of glycosylphosphatidylinositols (GPI-Aps) acting as membrane anchors of many eukaryotic cell surface proteins (Kinoshita T and Fujita M, 2016. J Lipid Res 57, 6-24).

### Cell Functions

PE plays an important role in several crucial cell functions such as cytokinesis (Emoto K and Umeda M, 2000. J Cell Biol 149, 1215-1224), and a lack of PE causes cell cycle arrest (Signorell A, et al., 2009. Trypanosoma brucei. Mol Microbiol 72, 1068-1079). PE also regulates the fusion of mitotic Golgi membranes (Pecheur E I, et al., 2002. Biochemistry 41, 9813-9823) and is the source of ethanolamine that is covalently bound to the eukaryotic elongation factor eEF1A (Signorell A, et al., 2008. J Biol Chem 283, 20320-20329). PE is required for the activity of several respiratory complexes supporting OXPHOS (Shinzawa-Itoh K, et al., 2007. EMBO J 26, 1713-1725; Tasseva G, et al., 2013, ibid). It plays a key role in autophagy via covalently binding to cytosolic LC3 to form membrane-bound LC3-II, which is recruited to autophagosomal membranes (Kabeya Y, et al., 2004, ibid; Rockenfeller P, et al., 2015, ibid). Moreover, mTOR inhibitors enhance both phospholipid re-modelling and autophagy that depends on PSD activity and mitochondrial PE (Thomas H E, et al., 2018. Cell Rep 24, 2404-2417, e2408). These PE functions may explain why SMAC depletion in tumors leads to exhaustion of cell PE while accumulating in the mitochondria, with pronounced decreased vesicle formation; altering nuclear morphology increased MAM; as well as inhibiting cell proliferation. Finally, PE plays a major role in cancer cell survival during metabolic stress conditions, (Zhu L and Bakovic M, 2012. Biochem Cell Biol 90, 188-199), implicated in ER stress relating to diabetes and neurodegeneration (Fu S, et al., 2011. 2011. Nature 473, 528-531). Furthermore, PE is associated with diseases, as its levels in substantia nigra pars compacta of Parkinson's disease patients is significantly lower compared with healthy subjects (Patel D and Witt S N, 2017, ibid).

### Nuclear Functions

The presence of chromatin-associated phospholipid and the role of intranuclear lipids have been reported in several studies (reviewed in Albi E and Viola Magni M P, 2004. Biol Cell 96, 657-667). Lipid metabolism in nuclei is very active and is involved in signal transduction into the nucleus in response to agonists acting at the plasma membrane level (D'Santos C S, et al., 1998. Biochim Biophys Acta 1436, 201-232). The presence of PL in chromatin and the nuclear matrix, and the roles of nuclear PL in the structural organization of chromatin and nucleic acid synthesis have been demonstrated (Alessenko and Burlakova, 2002, ibid). PL affects DNA and RNA metabolism (Shoji-Kawaguchi M, et al., 1995. J Biochem 117, 1095-1099; Albi E, et al., 1996. Cell Biol Int 20, 407-412; respectively), and PL content in the nuclei depends on the phase of DNA replication (Maraldi N M, et al., 1993, ibid). The present invention now demonstrates the presence of PE, SMAC and PSD, and their interacting/regulatory proteins, BIRC2, TRAF-2 and ARNT (Table 1) in the nucleus, thereby, regulating PE levels as a signaling molecule in the nucleus. As intranuclear PLs regulate DNA replication, upon SMAC depletion (described in Pual et al., 2021, ibid), changes in the expression of genes associated with the cell membrane, exosomes, ER- and Golgi-related proteins, and capillary organization may result from the decrease in phospholipid levels in the cell and, thus, in the nucleus. The nuclear localization of PSD and SMAC disclosed herein suggests a possible function of these proteins in the nucleus. Intranuclear lipids were found to function both in healthy and disease conditions (Albi and Viola, 2004, ibid) and numerous enzymes that synthesize and catabolize lipids were discovered within the nucleus (Ledeen L and Wu G, 2006. Nuclear lipids and their metabolic and signaling properties. In: Handbook of Neurochemistry and Molecular Neurobiology., pp. 173-198. Springer, Boston, MA). It has been previously shown by the Inventor of the present invention and co-workers that SMAC nuclear localization and its presence in the nucleus is a signature for squamous cell carcinoma (SCC), subtypes of non-small-cell lung cancer, as revealed by IHC and nuclear fractionation. The present invention shows for the first time that PSD is located in the nucleus; yet PE has been proposed to be present in the nucleus and its levels were increased in active chromatin (Albi and Viola, 2004, ibid).

In different tumors from patients (FIG. 15), both SMAC and PSD have been shown to be overexpressed and localized also in the nucleus, where PSD produces PE that regulates several nuclear properties and activities.

It is not clear what causes the translocation of PSD and SMAC into the nucleus, and their retention there most probably is mediated by binding to nucleus-located proteins. As demonstrated herein, PSD interacts with proteins also found in the nucleus, MAML2, HTRA2 and BIRC2. Thus, without wishing to be bound by any specific theory or mechanism of action, complex and dynamic interactions between PSD, SMAC and these proteins may take place in the nucleus, resulting in PE level modulation. Lipid is capable of regulating gene transcription, DNA replication or repair, and DNA cleavage, thereby regulating cell proliferation, differentiation, apoptosis and other cell functions (Faenza I, et al., 2013. FEBS J 280, 6311-6321). PLs and the enzyme precursors required for their synthesis were found in the nucleus as PIP2 (Keune W, et al., 2011. Adv Enzyme Regul 51, 91-99), polyphosphoinositide-metabolizing enzymes, kinases, phosphatases, phospholipases (Martelli A M, et al., 2011. Crit Rev Biochem Mol Biol 46, 436-457; Shah Z H, et al., 2013. FEBS J 280, 6295-6310) and PKC (Nishizuka Y, 1995. FASEB J 9, 484-496). Several metabolism-associated proteins were reported to be present in the nucleus including the mitochondrial pyruvate-dehydrogenase complex, generating locally acetyl-CoA to fuel histone acetylation (Sutendra G, et al., 2014. Cell 158, 84-97) or lipid biosynthesis (Chen J et al., 2018. Nat Genet 50, 219-228). Similarly, nuclear ATP-citrate lyase generating acetyl-CoA facilitating histone acetylation (Sivanand S, et al., 2017. Mol Cell 67,252-265.e256) acetyl-CoA synthetase-2 (Bulusu V, et al., 2017. Cell Rep 18, 647-658), thereby epigenetically regulating gene expression. Thus, nuclear localization of PSD, SMAC and some of their interacting proteins (BIRC2, MAML2, HTRA2), along with PE, point to the importance of PE production and its regulation of nuclear and cell functions (FIG. 17).

The present invention points to PSD as a target for cancer therapy, as demonstrated by the inhibition of its activity and the resulted inhibition in cell proliferation and tumor growth by novel PSD-interacting peptides. Rapidly growing tumor cells require not only an abundance of metabolites and nucleotides, but also PLs for membrane biogenesis and tumor development (Dobrzynska 1, et al., 2015, ibid). Limiting the availability of membrane lipids could be an efficient mechanism to inhibit dividing cells (van Meer G, et al., 2008. Nat Rev Mol Cell Biol 9, 112-124). The present invention provides compelling evidence that a mitochondrial phospholipid metabolism is linked to cell proliferation and tumor formation. Based on the differences that exist between the membranes of healthy cells and tumor cells, the present invention now discloses means for targeting membrane lipids in cancer cells as an anticancer drug. PE has been proposed as a target for anticancer therapy (Tan L T, et al., 2017. Front Pharmacol 8, 12) by small molecules such as duramycin and cinnamycin (Zhao M, 2011. Amino Acids 41, 1071-1079) and the natural product, ophiobolin A (OPA) (Chidley C, et al., 2016. Elife 5, e14601), which binds specifically to the PE molecules on the cancer cell membrane, subsequently leading to cell lysis (Tan et al., 2017, ibid).

The present invention now provides peptides capable of modulating PSD activity within the mitochondria and nuclei of mammalian cells. The only hitherto known inhibitor of PSD has been shown in malaria parasite (4-quinolinamine; Choi J Y, et al., 2016. Mol Microbiol 99, 999-1014).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Materials

Fluorescein isothiocyanate (FITC), hematoxylin, and eosin, PS, PE, sulforhodamine B (SRB), Triton X-100, and Tween-20 were obtained from Sigma (St. Louis, MO). Transfection agents JetPRIME and JetPEI were procured from PolyPlus transfection (Illkirch, France). Paraformaldehyde was obtained from Emsdiasum (Hatfield, PA). Dulbecco's modified Eagle's medium (DMEM) media was obtained from Gibco (Grand Island, NY). Normal goat serum (NGS) and the supplement fetal bovine serum (FBS), L-glutamine, and penicillin-streptomycin were obtained from Biological Industries (Beit Haemek, Israel). 3,3-diaminobenzidine (DAB) was obtained from Vector Laboratories (ImmPact-DAB, Burlingame, CA). Horseradish peroxidase (HRP)-conjugated secondary antibodies were procured from KPL (Gaithersburg, MD). Primary antibodies were obtained from different sources; their source, and dilutions are detailed in Table 1. Distyrylbenzene-bis-aldehyde (DSB-3) was synthesized in Prof. Uwe H. F. Bunz's group.

Tissue array sections (US Biomax BCN601) was obtained from US Biomax Inc., Derwood, MD). Human SMAC/Diablo-specific siRNA (si-hSMAC-A) was synthesized by Genepharma (Suzhou, China). Customized 768-peptide sequences derived from 11 SMAC-interacting proteins were arrayed on glass slides, produced by INTAVIS (Intavis Peptide Services, Tiibingen). Peptides were synthesized by GL Biochem (Shanghai, China) to >95% purity.

TABLE 1
Antibodies used in this study

| | | Dilution | |
|---|---|---|---|
| Antibody | Source and Cat. No. | IHC/IF | WB |
| Mouse monoclonal | Millipore, Billerica, MA, | — | 1:20000 |
| anti-actin | MAB1501 | | |
| Rabbit polyclonal | Abcam, ab8115 | 1:500 | 1:2000 |
| anti-SMAC/Diablo | | | |
| Rabbit monoclonal | Thermo Scientific, NY | 1:100 | — |
| anti-Ki-67 | RM9106-s1 | | |
| Mouse monoclonal | Abcam, Cambridge, UK, | 1:300 | 1:1000 |
| anti-ATP5a | ab14748 | | |
| Rabbit polyclonal | Abcam, Cambridge, UK | 1:200 | 1:4000 |
| anti-citrate synthase | ab96600 | | |
| Rabbit monoclonal | Abcam, Cambridge, UK, | 1:500 | 1:5000 |
| anti-VDAC1 | ab154856 | | |
| Mouse monoclonal | Abcam, Cambridge, UK, | 1:750 | 1:5000 |
| anti-VDAC1 | ab186321 | | |
| Mouse monoclonal | Abcam, Cambridge, UK, | — | 1:2000 |
| anti-GAPDH | ab9484 | | |
| Rabbit polyclonal | Abcam, Cambridge, UK, | — | 1:1000 |
| anti-H4 | ab10158 | | |
| Rabbit polyclonal | Biolegend, San Diego, USA, | 1:400 | — |
| anti-NFκB | Poly6226 | | |
| Mouse monoclonal | Santa Cruz, TX, sc-390070 | 1:300 | 1:2000 |
| anti-PSD | | | |
| Mouse monoclonal | Sigma, MO, HPA031090 | 1:500 | 1:3000 |
| anti-PSD | | | |
| Rabbit polyclonal | Abcam, Cambridge, UK, | 1:500 | |
| anti-IP3 receptor | ab5804 | | |
| Anti-Rabbit IgG, | Promega Corporation, | 1:1000 | 1:10000 |
| HRP conjugate | WI, USA | | |
| Anti-Mouse IgG, | Abcam, Cambridge, UK, | 1:1000 | 1:10000 |
| HRP conjugate | ab98799 | | |
| Anti-Mouse IgG, | Abcam, Cambridge, UK, | 1:750 | — |
| Alexa Fluor 488 | ab15010 | | |
| Anti-Mouse IgG, | Abcam, Cambridge, UK, | 1:750 | — |
| Alexa Fluor 555 | ab 15011 | | |
| Anti-Rabbit IgG, | Abcam, Cambridge, UK, | 1:750 | — |
| Alexa Fluor 555 | ab15008 | | |
| Anti-Rabbit IgG, | Thermo Fisher Scientific, | 1:750 | — |
| Alexa Fluor 488 | USA, A-11008 | | |

Table 1. Antibodies against the specific protein, source, catalogue number, and the dilutions used in immunohistochemistry (IHC), immunofluorescence (IF) and immunoblot (WB) are presented.

### Cell Culture and Transfection

A549 (human lung adenocarcinoma epithelial cell), HEK-293T (human embryonic kidney), H1563/CRL-5875 (non-small cell lung cancer adenocarcinoma), H-358 (bronchioalveolar carcinoma), HaCaT (spontaneously transformed aneuploid immortal human skin keratinocyte), and WI-38 (Caucasian fibroblast-like fetal lung cell) cell lines were purchased from the American Type Culture Collection (ATCC) (Manassas, VA), and were maintained as per ATCC instructions. Cells were maintained in ATCC recommended medium at 37° C. in an incubator with 5% CO₂. Cell lines were routinely tested for Mycoplasma contamination. For SMAC overexpression, cells were seeded (200,000 cells/well) on 6-well culture plates and allowed to adhere overnight. These adhered cells were transfected with PVMV3.1-SMAC or empty vector using the JetPRIME transfection reagent (Illkirch, France), according to the manufacturers' instructions. Medium was changed 6 h post transfection, and cells were harvested 48 h post transfection.

### SMAC/Diablo Gene Silencing

Human SMAC/Diablo-specific siRNA (si-hSMAC-A) sequences were:

Sense 5′AAGCGGUGUUUCUCAGAATTGtt3′ (SEQ ID NO:33) and anti-sense 5′AACAAUUCUGAGAAACCCGCtt3′ (SEQ ID NO:34). Cells were seeded (150,000 cells/well) in 6-well culture plates, cultured to 40-60% confluence, and transfected with 10-100 nM si-non-targeted (NT) or si-hSMAC-A using the JetPRIME transfection reagent, according to the manufacturer's instructions.

### Mouse Xenograft Experiment

Male athymic nude mice (6-8 weeks old) were obtained from Envigo and were kept in Ben-Gurion University (Israel) animal facility in temperature-controlled rooms and provided with water and food pellets ad libitum. Lung cancer A549 (3×10⁶) cells were implanted subcutaneously on the dorsal flanks. Tumor growth was recorded using digital calipers, and volumes were calculated using formula (π/6)*(L×W) (L=length; W=width). Mice were divided randomly into three groups once the average tumor volume reached ˜80 mm³, and were treated subcutaneously with si-NT (group 1) or si-hSMAC (group 2-350 nM group 3-700 nM final concentration) mixed with JetPEI reagent three times a week. Mice were sacrificed at the end of experiment, and the tumors were excised and processed for immunohistochemistry or frozen in liquid nitrogen for immunoblotting and RNA isolation. All procedures involving mice were approved by the Institutional Animal Care and Use Committee.

### PSD Protein Activity Inhibition

A549 cells were inoculated subcutaneously into female nude mice (7×10⁶ cells/mouse). Tumors sizes were measured (using a digital caliper) and volumes were calculated. When tumors volumes reached 60-100 mm3 (day 12), the mice were divided into 2 groups (control and treatment) and in the treatment group xenografts were treated with HtrA2/Omi-derived D-amino acid peptide (SEQ ID NO:35) targeted to the mitochondria. The tumors were injected 3 times a week to a final concentration of 50 μM or the peptide was administered by intravenous injection 2 times a week to a final concentration of 20 mg/kg. Tumors of A549 cell xenografts were dissected, photographed and weighted.

### CRISPR/Cas9 SMAC and PSD Knockout

SMAC CRISPR/Cas9 Knockout Plasmid with GFP marker (sc-402009) was purchased from Santa Cruz Biotechnology (Dallas, TX). A549 and HEK-293T cells were seeded in 6-well cell culture plates (200,000 cells/well) and allowed to attach overnight. Cells were transfected with SMAC CRISPR/Cas9 Knockout Plasmid as per manufacturers' instructions using JetPRIME transfection reagent. GFP-positive cells were sorted using FACS (SY3200 cell sorter—Synergy) and plated in 96-well plates (1 cell/well). Cells grew for 10 days, and each colony was transferred to a separate well of 12-well cell culture plates. Individual colonies having SMAC-KO were selected for maintenance after immunoblotting for SMAC. CRISPR/Cas9-mediated knockout of PSD was done in A549, HEK-293T, and NCI-H1563 (H1563/CRL-5875) cells by transfecting PSD CRISPR/Cas9 Knockout plasmid with GFP marker (sc-404398) as described for SMAC knockout, but no cells survived.

### Peptide Array

Customized 768-peptide sequences derived from 11 SMAC-interacting proteins produced by INTAVIS Peptide Services (GmbH & Co. KG, Tubingen, Germany) were arrayed on a glass slide. Peptide arrays were blotted with SMAC, PSD, or peptides interacting with PSD using anti-SMAC or anti-PSD antibodies. The interaction of purified SMAC or PSD with glass-bound peptide arrays was assayed following slide washing (3 times, 10 min each) with Tris-buffered saline (150 mM NaCl, 50 mM Tris-HCl, pH 7.4), followed by overnight incubation with blocking buffer (Tris-buffered saline containing low-fat dry milk, 2.5%, w/v). The slides were then incubated for 4 h or overnight with purified SMAC (0.3 or 0.8 μM) with and without pre-incubation with PSD (0.3 μM). Similarly, slides were incubated with PSD (0.15 μM) with or without pre-incubation with the indicated peptide (20 μM) in blocking buffer at room temperature. Following extensive slide washing with Tris-buffered saline containing 0.05% Tween-20, SMAC or PSD binding was detected using anti-SMAC or anti-PSD antibodies and HRP-conjugated anti-rabbit or anti-mouse IgG as a secondary antibody. The blots were developed using EZ-ECL (Biological Industries, Israel), according to the manufacturer's instructions.

### Immunohistochemistry (IHC) and Immunofluorescence (IF)

Formalin-fixed and paraffin-embedded tumor sections were deparaffinized using xylene and a series of ethanol treatments. Sections were then incubated with 3% H₂O₂ for 10 min to block endogenous peroxidase activity. Antigen retrieval was done in 0.01 M citrate buffer (pH 6.0) at 95-98° C. for 30 min, and washed with PBST (0.1% Tween 20). In order to reduce non-specific binding, sections were incubated in 10% normal goat serum for 2 h, then incubated with primary antibodies (Table 1) overnight at 4° C. For IHC, after washing with PBST, sections were incubated for 2 h at room temperature with HRP-conjugated secondary antibodies, washed well with PBST, and incubated with the substrate DAB. Sections were washed with water, counterstained with hematoxylin, and mounted with Vectashield mounting medium (Vector Laboratories, Burlingame, CA). Sections were observed under a microscope (Leica DM2500), and images were collected at 20× magnification with the same light intensity and exposure time. For IF, following overnight incubation with the primary antibodies, PBST-washed sections were incubated with fluorescent-tagged secondary antibodies for 2 h at room temperature in the dark. Following a wash with PBS, sections were incubated with DAPI for 15 min in the dark, washed, mounted with Vectashield mounting medium, and viewed by confocal microscopy (Olympus 1X81).

IF staining of cells was performed in cells plated on sterile glass coverslips placed in 12-well cell culture plates (30,000 cells/well) and incubated overnight in CO₂ incubator, washed with PBS, and fixed with 4% paraformaldehyde. Cells were then subjected to IF staining as described above for tissue sections.

### Protein Extraction and Immunoblotting

Cells were harvested and washed twice with ice-cold PBS, and the pellets were lysed on ice for 30 min in a lysis buffer (50 mM Tris-HCl, pH 7.5, 150 mM NaCl, 1 mM EDTA, 1.5 mM MgCl₂, 10% glycerol, 1% Triton X-100), freshly supplemented with a protease inhibitor cocktail (Calbiochem), incubated on ice for 20 min and centrifuged thereafter (10 min, 12000 g). The protein concentration of the supernatant was determined and cells were stored at −80° C. until used for gel electrophoresis and immunoblotting. Protein samples (10-20 μg) were subjected to SDS-PAGE and immunoblotting, using the selected primary antibodies, followed by incubation with HRP-conjugated secondary antibodies. HRP activity was determined using enhanced chemiluminescent substrate (Pierce Chemical, Rockford, IL). Band intensity was quantified using FUSION-FX (Vilber Lourmat, France).

### Sulforhodamine B (SRB) Cell Proliferation Assay

Cells were seeded in 96-well cell culture plates (8,000/well) and allowed to grow for 48 h. After washing with PBS, cells were fixed with 10% trichloroacetic acid, and stained with SRB for 20 min. Excess SRB were removed, and cells were washed with 1% acetic acid. SRB extraction was done using 100 mM Tris-base, and absorbance at 510 nm was determined using an Infinite M1000 plate reader (Tecan, Mannedorf, Switzerland).

### Cytoplasmic and Nuclear Protein Fractionation

A549 lung cancer cells were trypsinized and centrifuged at 600 g for 5 min. Cell pellet was washed with PBS, centrifuged at 600 g for 5 min, and cell pellet (2×10⁶ cells) was subjected to nuclear/cytosol fractionation using a Nuclear/Cytosol Fractionation Kit (K266-25; BioVision, Milpitas, CA), according to the manufacturer's instructions. Following centrifugation at 6,000×g for 10 min, the obtained supernatant (cytosolic fraction) and pellet (nuclear fraction) was re-suspended in the original volume. Samples were subjected to immunoblotting using anti-SMAC, anti-PSD, anti-ATP synthase 5a (mitochondria), anti-GAPDH (cytosol), and anti-H4 (nucleus) antibodies.

### Preparation of Mitochondria-Free, and Mitochondria-Enriched Fractions

A549 and A549-SMAC-KO cells (1.7×10⁸) were trypsinized and centrifuged at 600 g for 5 min, and cell pellet was washed with PBS and centrifuged at 600 g for 5 min. Cell pellet was re-suspended in 2.4 ml buffer (10 mM NaCl, 1.5 mM MgCl₂, and 10 mM Tris, pH 7.5), allowing for cell swelling for 10 min on ice. Cell suspensions were transferred to a glass homogenizer and subjected to homogenization using a tight pestle for 10 strokes. Six ml of mitochondrial suspension buffer (210 mM mannitol, 70 mM sucrose, 5 mM Tris-HCl, pH 7.5, 1 mM EDTA, pH 7.5) were mixed, and an additional 6 ml of the buffer was added to the suspension. Aliquots of the homogenate was kept (total extract), and the rest was centrifuged at 1300 g for 5 min and the supernatant was kept. This treatment was repeated twice, and the combined supernatant was centrifuged at 17,000 g for 10 min to obtain a pellet (mitochondria) and supernatant (mitochondria-free fraction). The pellet was resuspended in mitochondrial suspension buffer, and the protein amount was determined in all fractions.

### Determination of Total Phospholipids, PC and PR, and PSD Activity

Total lipids were extracted from the SMAC-KO and wild-type A549/HEK 293T cells by the Folch method (Folch J, et al., 1957. J Biol Chem 226, 497-509). Briefly, a cell suspension (2×10⁷ cells/ml) was made in CHCl₃/MeOH (2:1, v/v) and sonicated for 30 sec. Water (116 μl/ml) was added to the suspension and the suspension was shaken for 2 h at room temperature at 300 rpm. The aqueous suspension was heated for 10 min at 60° C., and stored at 4° C. for 3 h. Undissolved material was filtered through a 0.45-μm syringe filter. Solvent was evaporated under nitrogen, and residues were re-dissolved in CHCl₃/MeOH for lipid analysis.

Total phospholipid content of the cells was determined by colorimetric analysis at 488 nm based on the formation of a complex between the phospholipids and ammonium ferrothiocyanate. PC level was also determined by colorimetric analysis at 316 nm using ammonium thiocyanatocobaltate reagent, as described previously (Paul et al., 2018, ibid).

PE content in the lipid samples was measured as described previously (Choi J Y, et al. 2018. J Biol Chem 293, 1493-1503). Briefly, solvent was evaporated from the lipid samples under nitrogen gas, and samples were re-suspended in 0.8 mM Triton X-100. Sample aliquots were added to 96-well plates containing (50 μl) reaction mixture (80 mM NaCl, 2 mM K₂HPO₄, pH 7.4), and borate buffer (12.5 μl) (100 mM boric acid, 75 mM NaCl, 25 mM sodium tetraborate, pH 9). After mixing, 12.5 μl of 100 μM DSB-3, in 10 mM K₂HPO₄ (pH 7.4), were added and incubated for 2 h in the dark with shaking (100 rpm). Fluorescence was measured using excitation λex=403 nm and emission λem=508 nm. A calibration curve was obtained using PE (Biovision, CA) as a standard.

To determine PSD activity, purified enzyme (0.2-0.4 μM) or crude cell lysate (0.25-0.5 μg of protein) was incubated with PS (50 μM) for 45 min at 30° C. in the dark with shaking under the conditions described above for PE determination. PSD activity was measured by determining the PE produced as described above, except that the signal obtained in the absence of substrate PS (representing endogenous PE) was subtracted from the signal obtained in the presence of PS.

### DSB-3 PE Cell Staining

A549 and A549-SMAC-KO cells were seeded on sterile cover slips in 24-well cell culture plates (75,000 cells/well) and allowed to adhere for 24 h. MitoTracker® (250 nM in DMEM+10% FBS) was added, and plates were incubated in a CO₂ incubator for 30 min at 37° C. Cells were washed with PBS and incubated with DSB3 (10 μM in reaction buffer; 40 mM NaCl, 1 mM KPO4, pH 7.4) in a C02 incubator for 2 h. Cells were washed with PBS, fixed with 4% PFA for 20 min at room temperature, washed with PBS, and stained with DAPI for 20 min at room temperature. Cover slips were washed with PBS and mounted with Vectashield, and imaging was done using confocal microscopy (Olympus 1X81).

### Proximity Ligation Assay (PLA)

PLA was conducted as previously described (Gustafsdottir S M, et al. 2005. Analytical biochemistry 345, 2-9) using an in-situ detection reagent red kit (DuoLink InSitu PLA Probe kit, Sigma-Aldrich) according to the manufacturer's protocol. VDAC1-IP3R and SMAC-PSD were analyzed using PLA. Briefly, for tumor section, formalin-fixed and paraffin-embedded tumor sections (5-μm thick) were deparaffinized and then permeabilized using 0.3% Triton X-100 in PBS for 30 min, followed by blocking with Duolink® Blocking Solution for 1 h. Sections were incubated overnight with anti-VDAC1 (rabbit 1:750) and anti-IP3R (mouse, 1:750), washed twice with TBS-Tween (0.01%), and then incubated for 1 h with the secondary anti-mouse and anti-rabbit antibodies (PLA probe MINUS and PLUS) that were conjugated to complementary oligonucleotide extensions, and ligation and amplification steps were performed per manufacturer's instructions. The oligonucleotides hybridized with the subsequently added connector oligonucleotides, allowing formation of a circular DNA template. This circular DNA molecule was ligated and amplified, thereby creating a single-stranded DNA product covalently attached to one of the proximity probes, and hybridized Texas red-labeled oligonucleotide probes were detected.

For cells in culture, A549 cells and A549-SMAC-KO cells were seeded on 35-mm coverslips cultured to 60% confluency, washed with PBS, and fixed for 10 min at RT with fresh 4% formaldehyde solution in PBS, permeabilized with 0.1% Triton X-100 in PBS for 15 min at RT, washed once with PBS, and blocked for 30 min at 37° C. Blocking was carried out using Duolink Blocking Solution for 1 h. For PLA for SMAC and PSD close association, anti-SMAC (rabbit, 1:750) and anti-PSD (mouse, 1:750) were used followed by incubation with the secondary antibodies conjugated to the complementary oligonucleotide. Ligation and amplification steps were performed as described above for tumor sections. Cells were stained with DAPI and then mounted with round coverslips using aqueous mounting media with DAPI. Preparations were mounted in Vectashield mounting medium (Vector Laboratories, Burigame, CA, USA). Images were collected using a confocal microscope (Olympus TX81). Quantitation of protein levels, as reflected in the staining intensity, was analyzed in the whole area of the sections using Image J software.

### Purified PSD and SMAC

Human recombinant SMAC (10339-H08E) was produced by Sino Biologicals (Wayne, PA, USA). Purified recombinant protein (full length) of human phosphatidylserine decarboxylase (PSD), with N-terminal His tag, expressed in E. coli was obtained from Acris-OriGene.

### Micro-Scale Thermophoresis (MST)

MST analysis was performed using a NanoTemper Monolith NT.115 apparatus as described earlier (Wienken C J, et al., 2010. Nat Commun 1, 100). Briefly, purified SMAC purchased from Sino Biologicals (Wayne, PA) was fluorescently labeled using a NanoTemper Protein labeling kit BLUE (L001, NanoTemper Technologies). A constant concentration of SMAC was incubated with several concentrations of purified PSD in 10 mM Tris buffer, pH 7.4. After a 30-min incubation, 8 μl of the samples were loaded into a glass capillary (Monolith NT Capillaries), and thermophoresis was analyzed (LED 20%, IR laser 20%).

### Cell Penetration of Fluorescein Isothiocyanate (FITC)-Labeled Peptides

CPP-peptides (5 μM) were labeled with FITC by incubation for 30 min with 50 μM FITC in 10 mM Tricine buffer, pH 8.7, at 37° C. Unreacted reagent was removed by dialysis using membranes with a cutoff of 1000 Da (DiaEASY Dialyzer Floating Rack, BioVision, CA). A549 cells were incubated for 90 min with 5 μM FITC-labeled peptide in serum-free Dulbecco's modified Eagle's medium (DMEM), washed with PBS, fixed with 4% formaldehyde, and viewed under a confocal microscope (Olympus IX81).

### Statistics

Data are represented as means±SEM, and replicates are as indicated in figure legends. Numeric differences of interval level data between groups were compared using a two-tailed Student's t-test. Statistical significance is reported at p<0.05(*), p<0.01(**), or p<0.001(***).

### Example 1: SMAC/Diablo Depletion in Tumors or Cells in Culture-Inhibited Cell Proliferation

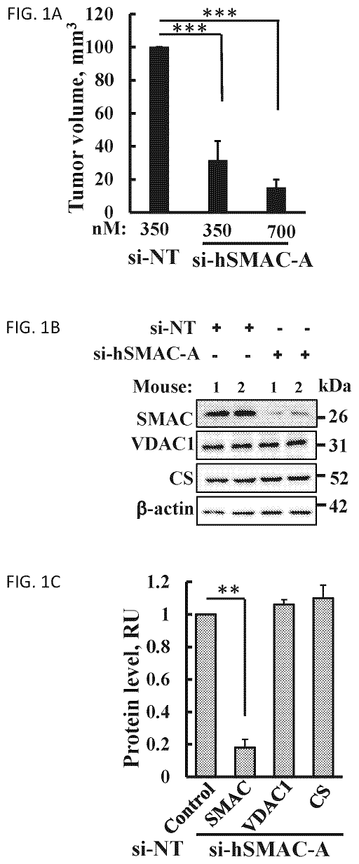

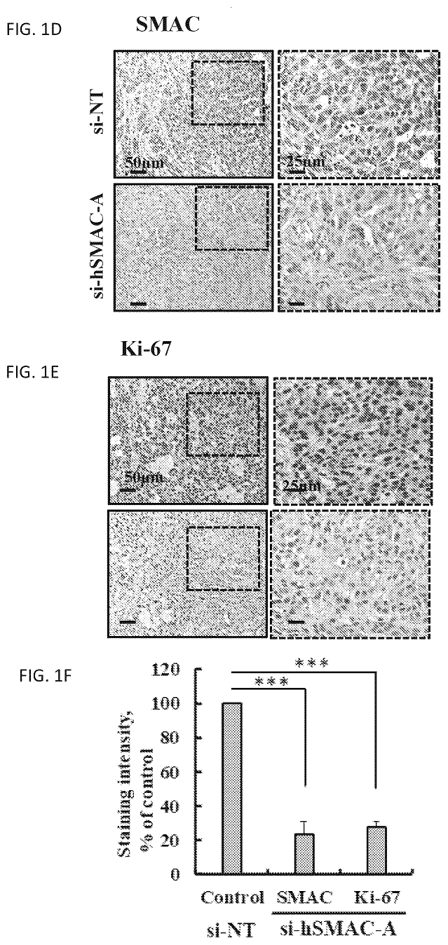

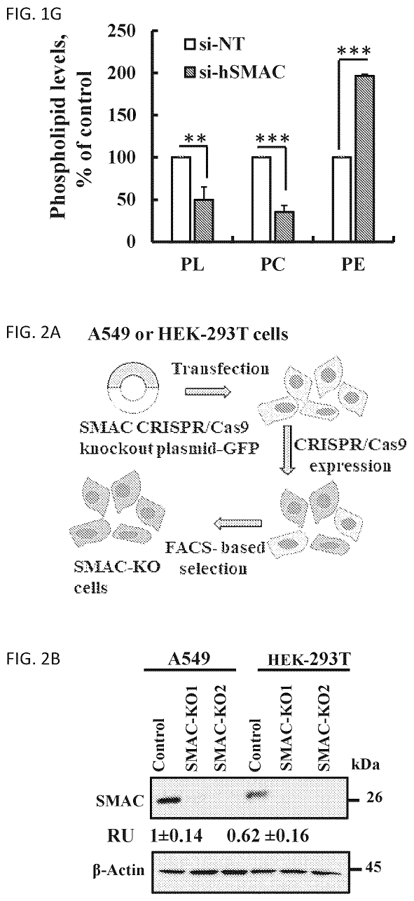

It has been previously demonstrated by the inventor and co-workers that silencing SMAC/Diablo expression using specific siRNA in sub-cutaneous xenografts of lung cancer A549 cells in mice reduced tumor growth (Paul et al., 2018, ibid; WO 2019/021,289). Similarly, tumor volume was reduced by 50% and 85% when treating mice with si-hSMAC-A at 350 nM and 700 nM, respectively, relative to the non-targeting siRNA (si-NT)-treated tumors (TTs) (FIG. 1A). Immunoblotting (FIG. 1B, C) and IHC (FIG. 1D-F) staining demonstrated that si-NT-TTs expressing high levels of SMAC/Diablo, that was decreased by 75% in si-hSMAC-A-TTs (FIG. 1D, F). The decrease in tumor volume is due to inhibition of cell proliferation as the expression of Ki-67, a cell proliferation factor, was highly reduced in the si-hSMAC-A-TTs (FIG. 1E, F). No cell death was detected in either si-NT-TTs or si-hSMAC-A-TTs, as revealed by TUNEL staining (data not shown).

The amounts of total phospholipids (PLs), PC, and PE were analyzed with the use of a relatively new PE probe DSB3 (Choi et al., 2018, ibid). Relative to si-NT-TTs, the PLs and PC were decreased by 50% and 65%, respectively, in si-hSMAC-A-TTs, while the levels of PE were increased by 200% (FIG. 1G).

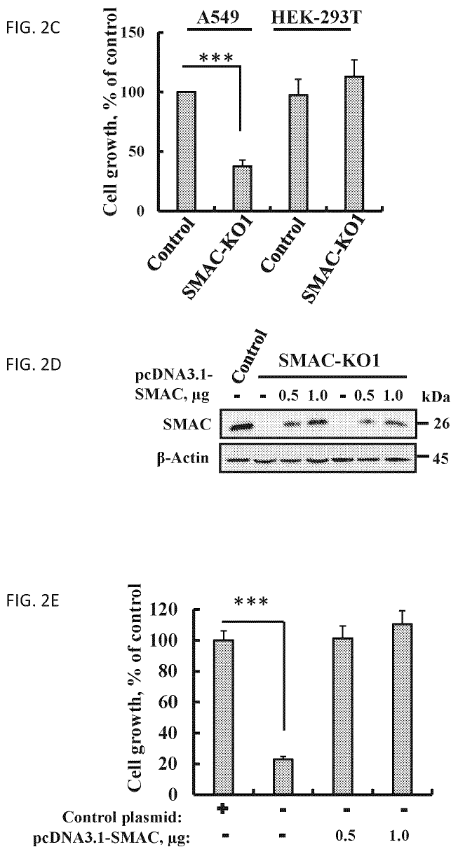

To validate the results obtained with the lung cancer xenograft, showing that their growth is highly dependent on SMAC and study SMAC mode of action, CRISPR/Cas9-mediated SMAC knockout (SMAC-KO) A549 lung cancer (A549:SMACA/A) and HEK-T-293 (HEK:SMACA/A) non-cancerous cell lines were generated (FIG. 2A). This allowed to test whether SMAC depletion confers different effects on cancer and non-cancerous cells. SMAC depletion in the A549:SMACA/A and HEK:SMACA/A cells was validated by immunofluorescence (IF) (not shown) and immunoblotting using anti-SMAC antibodies (FIG. 2B). The lung cancer A549:SMACA/A but not HEK:SMACA/A cells showed decreased cell growth (65%) as analyzed using the sulforhodamine B (SRB) assay (FIG. 2C).

To demonstrate that SMAC depletion is responsible for the inhibited cell growth, SMAC was re-expressed in the A549:SMACA/A cells using a SMAC-encoding plasmid. Re-expression of SMAC in these cells (FIG. 2D) restored cell growth (FIG. 2E), suggesting that the inhibited cell proliferation is directly related to SMAC cell depletion.

### Example 2: SMAC Depletion in Lung Tumors Increased ER-Mitochondria Contact Sites (MAM)

As previously shown, using transmission-electron microscopy (TEM), major alterations in the sub-cellular ultra-structures of si-hSMAC-A-TTs relative to that of si-NT-TTs were identified in the cells (Paul et al., 2018, ibid). In si-NT-TTs, a massive number of intracellular vesicles of different sizes and densities, such as large vesicles containing surfactant-accumulating lamellar bodies and others, was detected (FIG. 3A). Such vesicles were not observed in the si-hSMAC-A-TTs (FIG. 3B). In addition, in si-NT-TTs, the nuclear DNA was darkly stained representing heterochromatin, while in si-hSMAC-A-TTs, the nuclear DNA was found as euchromatin and not markedly stained (FIG. 3A, B).

As the major site of PL synthesis is ER-mitochondria contact sites (MAM) (Vance 2014, ibid), MAM-like structures in the EM images were searched. Interestingly, si-hSMAC-A-TTs sections were highly enriched in mitochondria (m) surrounded by ER (FIG. 3B e, f) that may represent MAMs.

To demonstrate the increase in ER-mitochondria interaction sites in the si-hSMAC-A-TTs, tissue sections were IF stained for the ER protein 1,4,5-trisphosphate receptor (IP3R) and for VDAC1 as outer mitochondrial membrane (OMM) protein using specific antibodies (FIG. 3C). The IF results clearly indicate that both IP₃R and VDAC1 staining levels in si-hSMAC-A-TTs were highly increased relative to their staining in si-NT-TTs (FIG. 3C).

Next, to validate that the increase in ER surrounding mitochondria (FIG. 3B) represents MAM, in-situ proximity ligation assay (PLA) was performed (FIG. 3D). This enables visualizing protein-protein interactions (˜0-40 nm) reporting molecular recognition (Soderberg 0, et al. 2006. Nat Methods 3, 995-1000). PLA assay using anti-VDAC1 and anti-IP3R antibodies showed a strong signal in the si-hSMAC-A-TTs sections relative to that observed in si-NT-TTs, suggesting an increase in MAM (FIG. 3D), confirming the close proximity of VDAC1 at the mitochondria and IP₃R at the ER, both components of the MAM (Vance 2014, ibid). No signal was obtained in PLA using SMAC and the inner mitochondria membrane (TMM) protein, ATP synthase subunit 5A antibodies (data not shown).

### Example 3: SMAC Cell Depletion Regulates Phospholipid Synthesis Via Modulating the Mitochondrial PSD in Cancer Cells

SMAC expressing- and SMAC-KO A549 cells represent comparative models to investigate SMAC function in regulating phospholipid synthesis. Thus, the levels of PL, PC, and PE in the A549-SMAC-KO- and HEK-293T-KO cells relative to the same cell lines expressing SMAC were analyzed. PE levels were assayed using a specific probe, DSB3 (Choi et al., 2018, ibid) (FIG. 4A). In the lung cancer A549-SMAC-KO cells, the levels of PLs and PC were reduced (2-fold), while the level of PE was increased 2-fold (FIG. 4B). HEK-293T-SMAC-KO and SMAC expressing cells, however, showed similar levels of PL, PC, and PE (FIG. 4B). Interestingly, the cell size of the A549-SMAC-KO was increased (data not shown), which may suggest changes in membrane fluidity.

One of the key proteins in PE synthesis is PSD, a mitochondrial enzyme localized as SMAC in the IMS. PSD catalyzes the formation of PE from PS transported from the ER to the mitochondria (FIG. 4C). Therefore, we asked whether the increase in PE obtained in the lung cancer A549 SMAC-KO cells was due to increased PSD activity owing to the absence of SMAC. Accordingly, we analyzed in A549-SMAC-KO cells the PSD activity and PE levels that were increased about 2-fold relative to SMAC-expressing A549 cells, while HEK-293T SMAC expressing- or SMAC-KO cells showed similar PSD activity (FIG. 4D) and PE levels (FIG. 4E). These results indicate that upon SMAC-KO, PSD activity and PE levels were increased in cancer cells, but not in the tested non-tumorigenic cell line. It should be indicated that HEK-293 can be tumorigenic (Arbiser J L, et al., 1999. Invest Dermatol. 113(5), 838-842).

Next it was examined whether PE content in the mitochondria of SMAC-KO cells is increased due to activation of PSD converting PS, produced in the ER from PC and PE by PS-synthase 1 (PSS1) and PSS2, respectively (Vance J E. 2008. J Lipid Res 49, 1377-1387), into PE (FIG. 4C). Mitochondria-enriched and mitochondria-free fractions were obtained from control and SMAC-KO A549 cells, as validated by the presence of ATP synthase subunit 5a (ATPsyn5a), an inner mitochondria membrane (IMM) protein absent from the mitochondria-free fraction (FIG. 4F). SMAC is enriched in the control mitochondria fraction and, as expected, lacking in the SMAC-KO cells, while PSD is found in both mitochondria-free and mitochondria-enriched fractions (FIG. 4F). These fractions were analyzed for PSD activity, showing increased activity in SMAC-KO cell extract, as well as in the mitochondrial fraction derived from control and further increased in SMAC-KO cells (FIG. 4G, Table 2).

The levels of PLs, PC, and PE in the total phospholipid extract in these fractions were also analyzed (FIG. 4H). As found for the si-hSMAC-TTs (FIG. 1G) and SMAC-KO cells (FIG. 4B), the levels of PLs and PC were decreased about 2-fold, while PE level was over 2-fold higher in the total cell phospholipid extract and in the mitochondria-enriched fraction, but not in the mitochondria-free fraction (FIG. 4H). The increase in PE levels in the SMAC-KO cells (FIG. 4B) and tumors (FIG. 1G) suggests that upon SMAC depletion, PSD is activated, leading to increased PE production in the mitochondria at the expense of PC, PS, and PE in the ER.

TABLE 2
Analysis of PL, PC, and PE levels in cell extract, mitochondria-free,
and mitochondria-enriched fractions obtained from A549 cells
expression or KO for SMAC

| | | Phospholipid level, nmole/mg of protein | | |
|---|---|---|---|---|
| No | Fraction | PL | PC | PE |
| 1 | A549 total | 74.62 ± 6.22 | 44.64 ± 2.06 | 37.37 ± 2.21 |
| | cells extract | | | |
| 2 | A549 | 29.80 ± 1.12 | 15.36 ± 0.03 | 24.52 ± 1.0 |
| | mitochondria-free | | | |
| 3 | A549 | 100.17 ± 8.55 | 62.60 ± 1.0 | 69.33 ± 12.56 |
| | mitochondria | | | |
| 4 | A549-KO total | 39.55 ± 2.88 | 21.35 ± 0.55 | 7.12 ± 13.6 |
| | cells extract | | | |
| 5 | A549-KO | 15.98 ± 0.13 | 8.98 ± 0.07 | 24.47 ± 5.88 |
| | mitochondria-free | | | |
| 6 | A549-KO- | 52.5 ± 4.69 | 32.6 ± 0.67 | 143.19 ± 8.89 |
| | mitochondria | | | |

### Example 4: PE-Specific Probe DSB-3 can be Used to Unravel PE Levels in Sub-Cellular Compartments

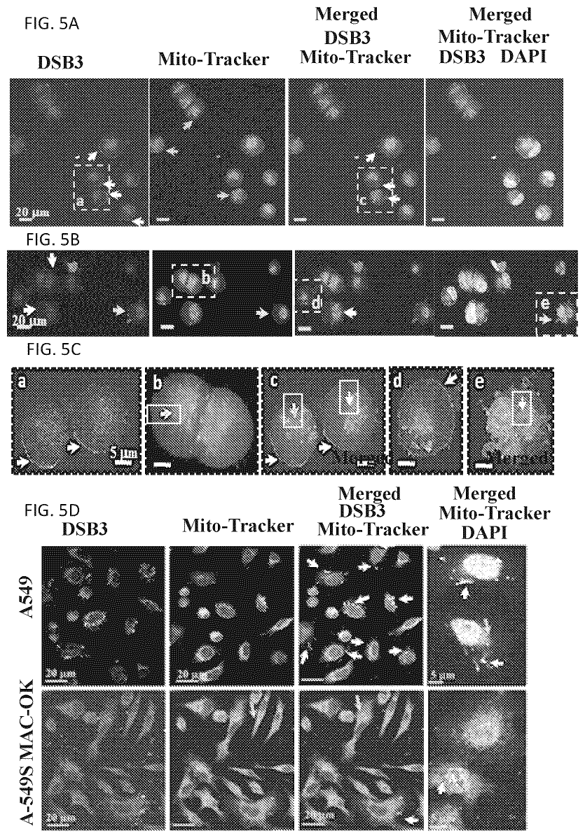

The PE-specific probe DSB-3, used to assay PE levels in solution is used here, for the first time, to unravel PE levels in sub-cellular compartments in A-549 cells, using confocal fluorescence imaging (FIG. 5). Even though DSB-3 is a small molecule probe, and PE is present in most membranes, the results showed that PE was enriched in the mitochondria. The results clearly show green staining representing DSB-3 labeled PE at the plasma membrane (arrows, A a, c, d), and in other cell compartments, possibly ER. In addition, all green fluorescence was co-localized with MitoTracker, (FIG. 5A-C), suggesting that most PE is in the mitochondria, in agreement with the enrichment of PE in the mitochondrial fraction of SMAC-KO cells (FIG. 5D). An apoptotic cell with membrane blebbing is also shown (FIG. 5C e).

PE-specific probes, duramycin and cinnamycin, that bind with high affinity to the head group of PE were previously developed (Zhao M. 2011. Amino Acids 41, 1071-1079). However, these reagents affect membrane reorganization and permeabilization, eventually leading to cell death (Tan L T, et al. 2017. Front Pharmacol 8, 12). DSB-3 on the other hand induces no cell death (data not shown) and, thus, may assist in unraveling PE levels and sub-cellular localization under different conditions.

### Example 5: SMAC and PSD Expression Levels in Cancer and Non-Cancerous Cell Lines and SMAC Interaction with and Inhibition of PSD Activity

SMAC and PSD are both located in the IMS, as also demonstrated here by IF staining (FIG. 6A). The close association of SMAC with PSD is demonstrated by in-situ PLA assay, with a signal obtained in SMAC-expressing, but not in SMAC-KO A549 cells (FIG. 6B), indicating close proximity of the two proteins, and pointing to a specific signal being obtained only when the two proteins are present. The PLA signal is specific, as no signal was obtained when PLA was performed using PSD and citrate synthase (CS) located in the matrix (data not shown). PLA was previously used to demonstrate interaction between proteins such as Myc and Max in response IFN-γ signaling (Soderberg et al., 2006, ibid).

The relationship between SMAC and PSD were evaluated by analyzing their expression levels (FIG. 6C D), PSD activity (FIG. 6E), and PE levels (FIG. 6F) in different cell lines, cancerous (A549, H3538, NCI-H-1563) and non-cancerous cells (Wi-38, HEK-293T, HaCaT). The results indicate that both proteins were highly expressed (2- to 7-fold) in cancerous cells relative to non-cancerous cells, with no significant difference in the expression of the mitochondrial protein, citrate synthase (FIG. 6C, D). Similar results were obtained when PSD activity (FIG. 6E) or PE levels (FIG. 6F) were analyzed. The results show a correlation between SMAC and PSD expression level, PSD activity, and PE levels with NCI-H-1563 showing the highest values.

The direct interaction of purified SMAC with purified PSD was demonstrated using the microscale thermophoresis (MST) (FIG. 6G). PSD interacts with SMAC with a high affinity with a dissociation constant of 246 nM (FIG. 6H). SMAC interaction with PSD also is reflected in SMAC-inhibiting PSD activity with IC₅₀ of 400 nM (FIG. 6I), in agreement with the increase in PE levels in si-hSMAC-TTs (FIG. 1G) and in CRISPER/Cas9-KO cells (FIG. 4E).

### Example 6: Identification of SMAC-PSD Interaction Site Using Peptide Arrays Composed of Peptides Derived from SMAC-Interacting Proteins

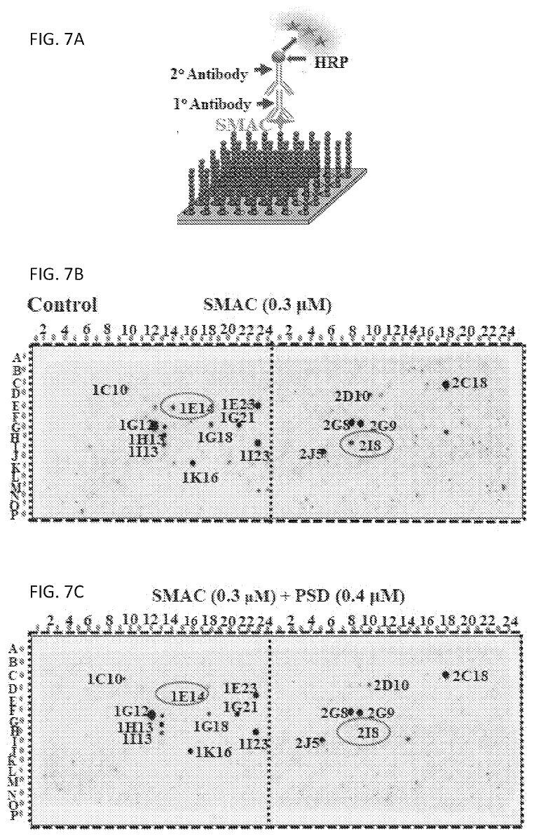

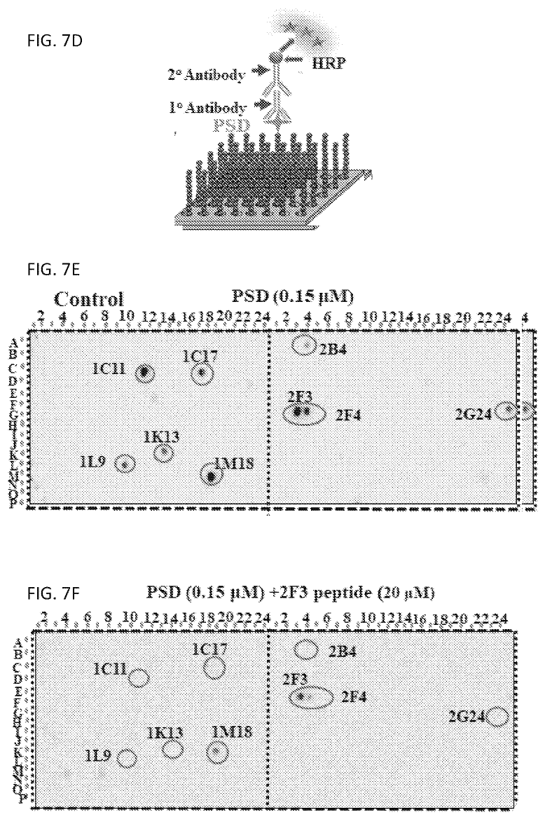

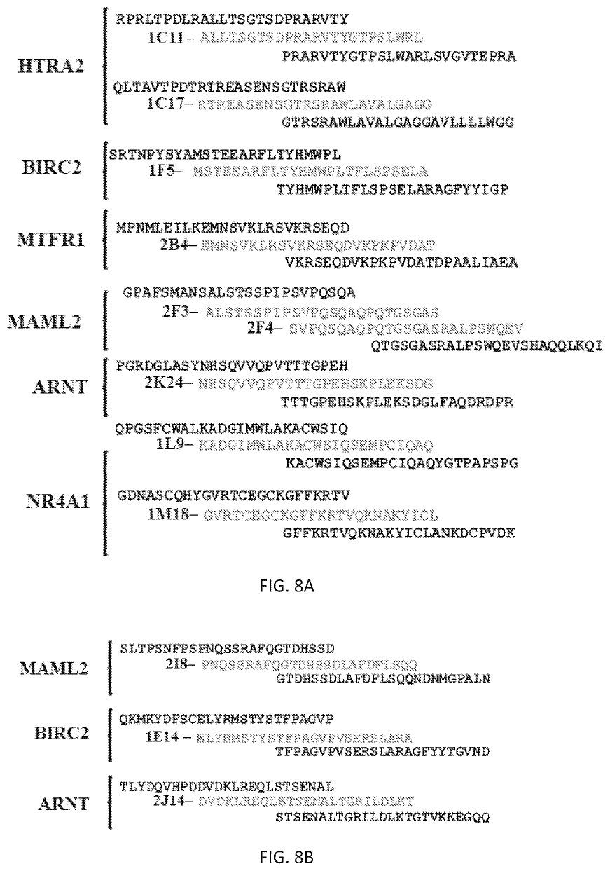

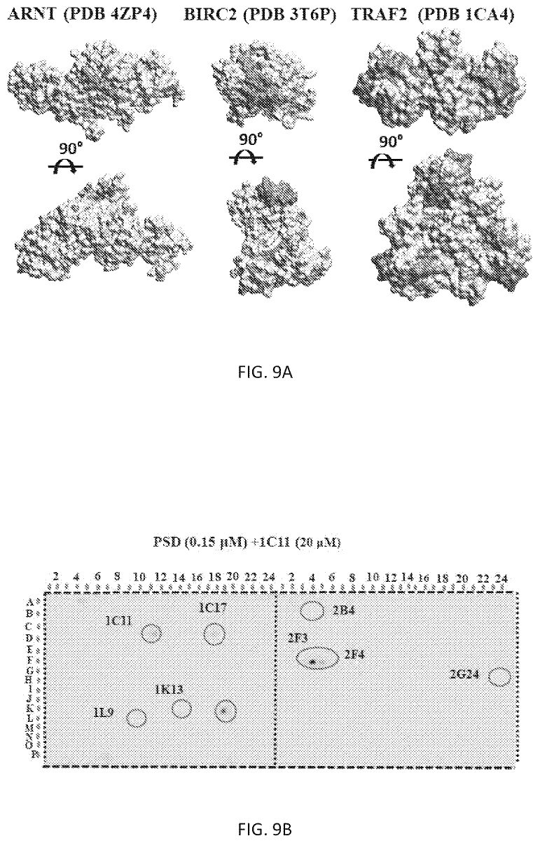

To identify possible SMAC-PSD interaction site(s), glass-bound peptide arrays were designed, consisting of 768 overlapping peptides derived from 15 selected SMAC-interacting proteins (reported and identified using the string link program) and tested whether PSD interferes with SMAC interaction with any peptide in the array. The array was blotted with purified SMAC and then with anti-SMAC antibodies (FIG. 7A), showing the binding of SMAC to 16 peptides derived from nine proteins (FIG. 7B). Pre-incubation of SMAC with purified PSD prevented SMAC interaction with three peptide spots: 1E14, a peptide derived from the protein BIRC2 (baculoviral IAP2 repeat containing 2); peptide 218, derived from MAML2 (mastermind like transcriptional coactivator 2 truncated poly Q); and peptide 2J14, derived from ARNT (aryl hydrocarbon receptor nuclear translocator isoform 1, also known as the HIF-1β subunit (FIGS. 7C, and 8, Table 3). Using available structures for BIRC, MAML2, and ARNT proteins, the sequences interacting with PSD were found to be localized on the protein surface, thus, exposed to interaction with PSD (FIG. 9A). Without wishing to be bound by any specific theory or mechanism of action, this may suggest that the interaction of PSD with SMAC prevented SMAC interaction with these three peptides derived from three different proteins; alternatively, PSD may interact with these peptides in the array and prevents SMAC binding to them. To test this, the peptide array was blotted with purified PSD followed by anti-PSD antibodies. It was found that PSD interacted with nine peptides derived from six different proteins (FIG. 7D, E, FIG. 9), the IMS protein HTRA2 (serine peptidase 2), TRAF-2 (TNF receptor associated factor 2), MTFR1 (mitochondrial fission regulator 1), MAML2, ARNT, and BIRC2. Interestingly, PSD prevented MAML2, ARNT, and BIRC2 binding to SMAC (FIG. 7C, Table 3). However, the peptide interacted with PSD through motifs that are not involved in SMAC binding. Remarkably, these three proteins are located in the nucleus (Samuel T, et al., 2005. Cancer Res 65, 210-218; Seok S H, et al., 2017. Proc Natl Acad Sci USA 114, 5431-5436). The presence of SMAC and PSD in the nucleus is shown hereinbelow.

The peptides, 2F3, derived from the nuclear protein MAML2 and IC11, derived from inter mitochondrial membrane space (IMS) protein HTRA2 showing the highest interaction with PSD (as reflected in the spot intensity, FIG. 7E) were synthesized and when pre-incubated with PSD, they eliminate PSD interaction with most peptides (FIGS. 7F, FIG. 9B), pointing to PSD-specific interaction.

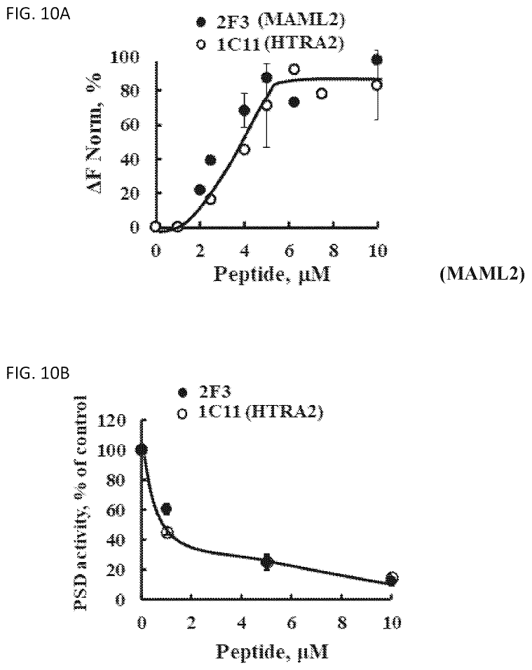

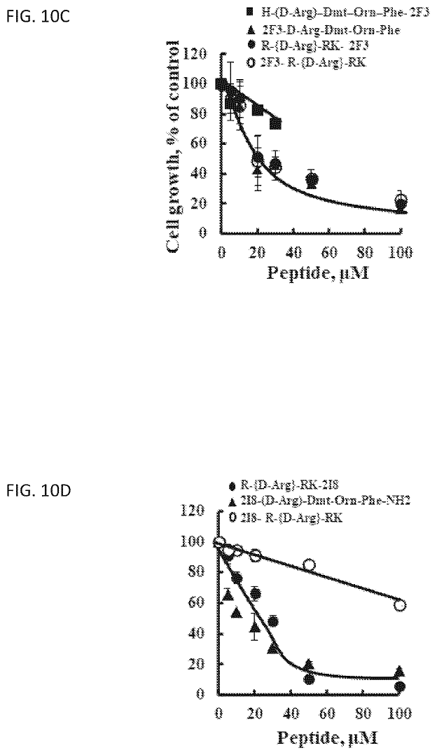

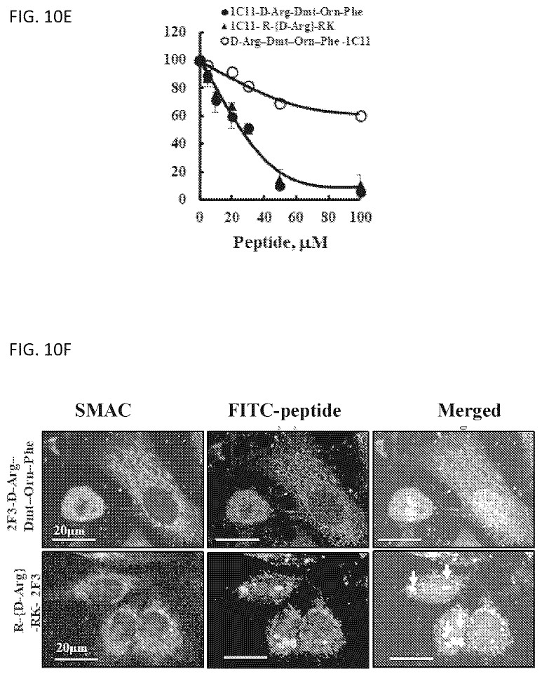

Purified PSD interacted with both synthetic peptides with a similar binding affinity (Kd of 3 μM), as monitored using MST (FIG. 10A) and inhibition of PSD activity with IC₅₀ of 1 μM (FIG. 10B), suggesting specific interactions.

TABLE 3
Proteins identified as interacting with PSD or
their interaction with is prevented in the
SMAC/Diablo presence of PSD

| | | B. Identified |
|---|---|---|
| | A. Identified | by PSD inhibiting |
| | by blotting | SMAC binding, |
| | with PSD Spot | Spot No, |
| Protein | No, Sequence | Sequence |
| HTRA2 | 1C11- | |
| | ALLTSGTSDPRARVTY | |
| | GTPSLWARL | |
| | (SEQ ID NO: 1) | |
| | 1C17- | |
| | RTREASENSGTRSRAW | |
| | LAVALGAGG | |
| | (SEQ ID NO: 14) | |
| BIRC2 | 1F5- | 1E14- |
| | MSTEEARFLTYHMWPL | FQQLTCVLPILDNLLKANV |
| | TFLSPSELA | INKQEH |
| | (SEQ ID NO: 2) | (SEQ ID NO: 3) |
| TRAF-2 | 1K13- | |
| | EMEASTYDGFIWKISD | |
| | FARKRQE | |
| | (SEQ ID NO: 13) | |
| MTFR1 | 2B4- | |
| | EMNSVKLRSVKRSEQD | |
| | VKPKPVDAT | |
| | (SEQ ID NO: 8) | |
| MAML2 | 2F3- | 218- |
| | ALSTSSPIPSVPQSQA | PNQSSRAFQGTDHSSDLAF |
| | QPQTGSGAS | DFLSQQ |
| | (SEQ ID NO: 4) | (SEQ ID NO: 7) |
| | 2F4- | |
| | SVPQSQAQPQTGSGAS | |
| | RALPSWQEV | |
| | (SEQ ID NO: 5) | |
| | 2G24- | |
| | KDQRRNVGNMQPTAQY | |
| | SGSSTISL | |
| | (SEQ ID NO: 6) | |
| ARNT | 2K24- | 2J14- |
| | NHSQVVQPVTTTGPEH | DVDKLREQLSTSENALTGR |
| | SKPLEKSDG | ILDLKT |
| | (SEQ ID NO: 9) | (SEQ ID NO: 10) |
| NR4A1 | 1L9- | |
| | KADGIMWLAKACWSIQ | |
| | SEMPCIQAQ | |
| | (SEQ ID NO: 11) | |
| | 1M18- | |
| | GVRTCEGCKGFFKRTV | |
| | QKNAKYICL | |
| | (SEQ ID NO: 12) | |

### Example 7: Cell-Penetrating PSD-Interacting Peptides Targeted to Mitochondria or the Nucleus Inhibit Cell Proliferation

The peptides 2F3 and IC11, derived from MAML2 and HTRA2, respectively, and peptide 218, derived from MAML2, but identified by PSD inhibiting SMAC binding to it, were designed as cell-penetrating peptides (CPPs) targeted to mitochondria or the nucleus and tested for their effects on cell proliferation (FIG. 10C-E, Table 4). To target the peptide to the nucleus (nuCPP), the peptides were fused to the tetrapeptide RrRK (r=D-arginine, SEQ ID NO:16), shown to primarily target into the nucleus of HeLa cells (Puckett C A, et al., 2010. Bioorg Med Chem 18, 3564-3569). To target the peptides to mitochondria, the peptides were fused to a mitochondria-targeting sequence (mtCPP-1) composed of D-Arg-Dmt-Orn-Phe, where Dmt is 2,6-dimethyl-L-tyrosine (SEQ ID NO:15), optionally further comprising amid group (NH₂) at the C-terminus. mtCPP-1 displayed at least three characteristics known to be important for mitochondrial targeting: positive charge, lipophilicity, and alternating aromatic and basic residues. mtCPP-1 showed no toxicity in the different cell lines, and did not induce apoptosis (Cerrato C P, et al., 2015. FASEB J 29, 4589-4599).

The mtCPP-2F3 peptide, derived from the nuclear protein MAML2, inhibited the growth of A549 cancer cells when the mitochondrial targeting sequence was located at the C-terminus of the peptide, (IC₅₀, 19.6 μM), but no inhibition (IC₅₀, >100 μM) was obtained when mtCPP was localized at the N-terminus of the peptide (FIG. 10C, Table 4). In contrast, nuCPP added to the N- or the C-terminus of 2F3 peptide resulted in similar cell proliferation inhibition (IC₅₀, 19.6 μM). Another peptide, 218, derived from MAML2, inhibited cell proliferation when the mitochondrial- or nuclear-targeting sequence was fused at the C-terminus, with IC50 of 19.7 and 30 μM for the nuclear and mitochondrial targeting, respectively, but no significant inhibition was obtained when the mitochondrial- or nuclear-targeting sequence was at the N-terminus (IC₅₀, >100 and 67 μM, respectively) (FIG. 10D, Table 4).

Similar results were obtained with CPP-IC11, inhibiting cell growth (IC₅₀, 30 μM) when the targeting sequence to the mitochondria or to the nucleus was localized to the C-terminus, but not fused to the N-terminus of the peptide (FIG. 10E, Table 4).

Interestingly, peptide 1E14 derived from BIRC2 (mainly located to the cytosol) and identified by PSD-preventing SMAC interaction, had no effect on cell growth when targeted to the mitochondria or the nucleus (Table 4).

To demonstrate that the peptides reach the mitochondria or the nucleus, they were FITC-labeled (FIG. 10F). Confocal images clearly show that the peptides were localized to the mitochondria or the nucleus, according to their targeting sequence. The nuclear-targeted peptides also reached the mitochondria, most probably due to their high positive charge. Thus, peptides interacting with PSD inhibited cell proliferation when targeted to the mitochondria or nucleus.

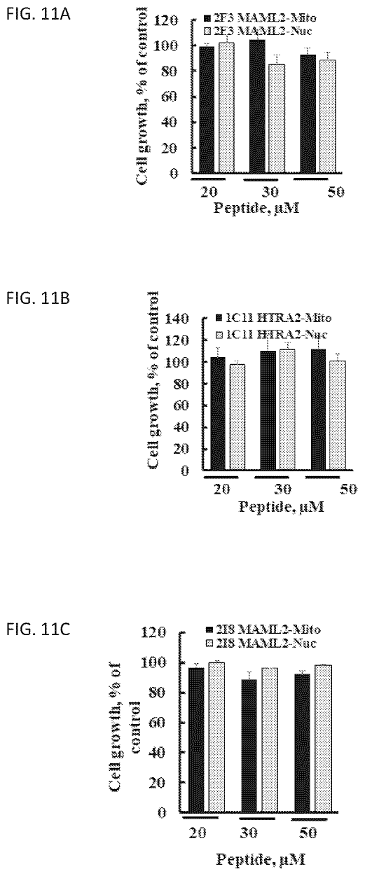

Selected peptides were tested for their effect on non-cancerous HaCaT cells showing no significant inhibition on cell proliferation (FIG. 11).

TABLE 4
Effects of peptide identified as interacting with PSD or their
interaction with SMAC/Diablo is prevented in the presence of
PSD on A549 lung cancer cell growth

| Peptide | | | |
|---|---|---|---|
| Spot | | | |
| SEQ ID | | Peptide and Cellular | |
| No. | Protein | Targeting | IC50 μM |
| 2F3 | MAML2 | 2F3-D-Arg-Dmt-Orn-Phe | 19.6 ± 0.4 |
| SEQ ID | | Mitochondria (A) | |
| NO: 4 | | (SEQ ID NO: 17) | |
| Interacting | | H-(D-Arg)-Dmt-Orn-Phe-2F3 | >100 |
| with PSD | | Mitochondria | |
| | | (SEQ ID NO: 18) | |
| | | R-{D-Arg}-RK-2F3 | 28.9 ± 5.2 |
| | | Nucleus (SEQ ID NO: 19) | |
| | | 2F3-R-{D-Arg}-RK | 29.6 ± 0.6 |
| | | Nucleus (SEQ ID NO: 20) | |
| 2I8 | MAML2 | 2I8-(D-Arg)-Dmt-Orn- | 30.2 ± 3.6 |
| SEQ ID | | Phe-NH2 Mitochondria | |
| NO: 7 | | (SEQ ID NO: 21) | |
| Identified | | D-Arg-Dmt-Orn-Phe-2I8 | 66.8 ± 4.7 |
| by PSD | | Mitochondria | |
| preventing | | (SEQ ID NO: 22) | |
| SMAC | | R-{D-Arg}-RK-2I8 | >100 |
| interaction | | Nucleus (SEQ ID NO: 23) | |
| with it | | 2I8-R-{D-Arg}-RK | 19.7 ± 4.3 |
| | | Nucleus (SEQ ID NO: 24) | |
| 1C11 | HTRA2 | 1C11-D-Arg-Dmt-Orn-Phe | 30.3 ± 5.5 |
| SEQ ID | | Mitochondria | |
| NO: 1 | | (SEQ ID NO: 25) | |
| Interacting | | D-Arg-Dmt-Orn-Phe-1C11 | 89.3 ± 5.49 |
| with PSD | | Mitochondria | |
| | | (SEQ ID NO: 26) | |
| | | R-{D-Arg}-RK-1C11 | 66.5 ± 3.9 |
| | | Nucleus (SEQ ID NO: 27) | |
| | | 1C11-R-{D-Arg}-RK | 30.0 ± 3.8 |
| | | Nucleus (SEQ ID NO: 28) | |
| 1E14 | BIRC2 | D-Arg-Dmt-Orn-Phe-1E14 | >100 |
| SEQ ID | (Baculoviral- | Mitochondria | |
| NO: 3 | IAP2 repeat) | (SEQ ID NO: 29) | |
| Identified | | | |
| by PSD | | | |
| preventing | | | |
| SMAC | | | |
| interaction | | | |
| with it | | | |
| | | 1E14-D-Arg-Dmt-Orn-Phe | >100 |
| | | Mitochondria | |
| | | (SEQ ID NO: 30) | |
| | | R-{D-Arg}-RK-1E14 | >100 |
| | | Nucleus (SEQ ID NO: 31) | |

### Example 8: Activity of Modified PSD-Interacting Peptides

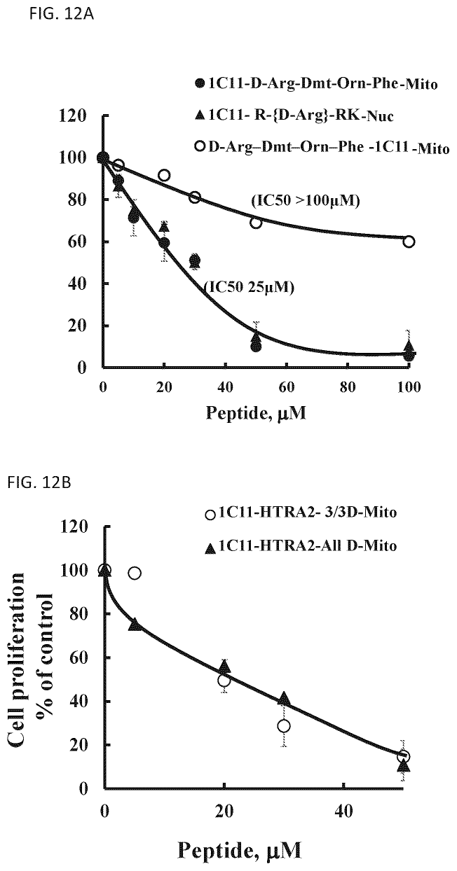

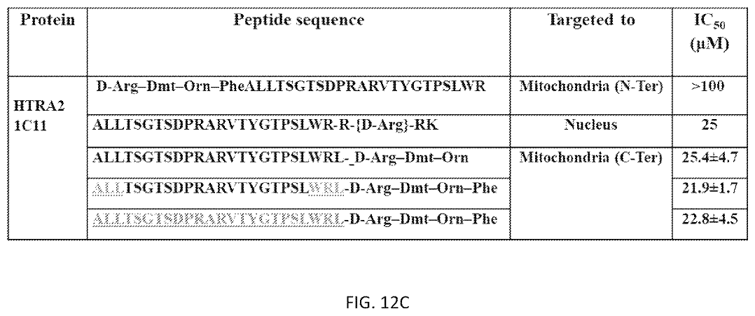

To increase stability of the identified PSD-interacting peptides, L-amino acids were replaced with D-amino acid in several positions. The effect of the amino acid configuration change on cell proliferation compared to unmodified peptides was examined (FIG. 12 B, C). The results show that modifying the HTRA2-derived peptide 1C11 with all or only three amino acids in C- and N-terminus with the D-configuration showed similar cell proliferation inhibition (FIG. 12A, B, C). Modified D-peptides are expected to be more stable to protease degradation and to exhibit less immunogenicity.

Upon apoptotic signal, HTRA2 (HtrA2/Omni is released into the cytoplasm and binds inhibitor of apoptosis proteins (IAPs), thus promoting the activation of caspases and initiates apoptosis (Yang Q H, et al., 2003. Genes Dev 17(12) 1487-96). It is also proposed that upon external signals, such as high temperature or hypoxia this protein is translocated to the mitochondrial matrix where it regulates cell death by modulating apoptotic and autophagic pathways (Blink E, et al., 2004. Cell Death Differ 11(8) 937-939); Gieldon A, et al., 2016. PLoS One 11(8) e0161526).

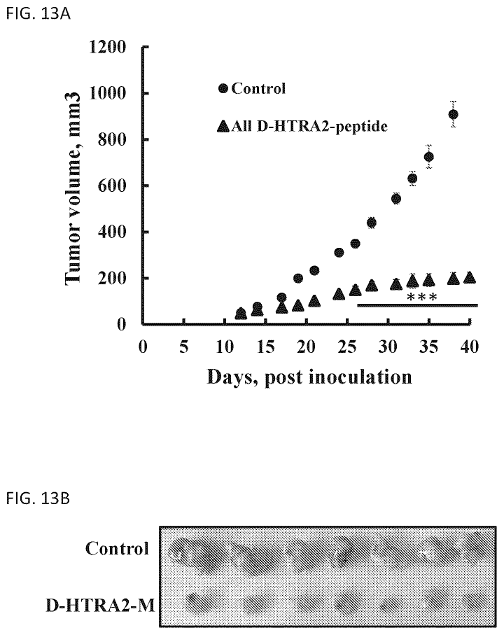

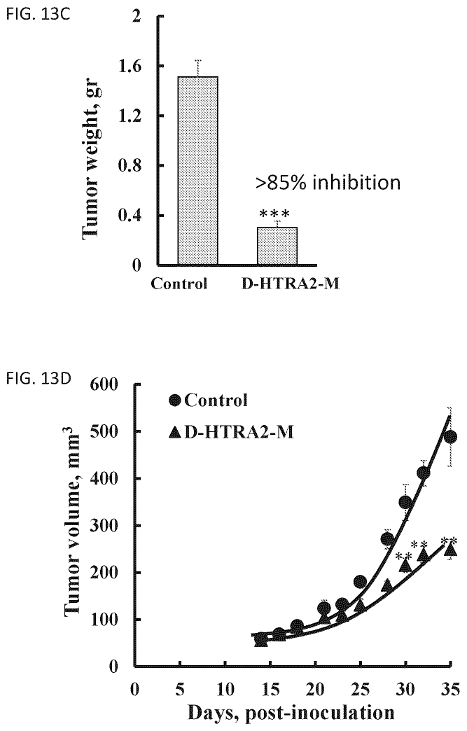

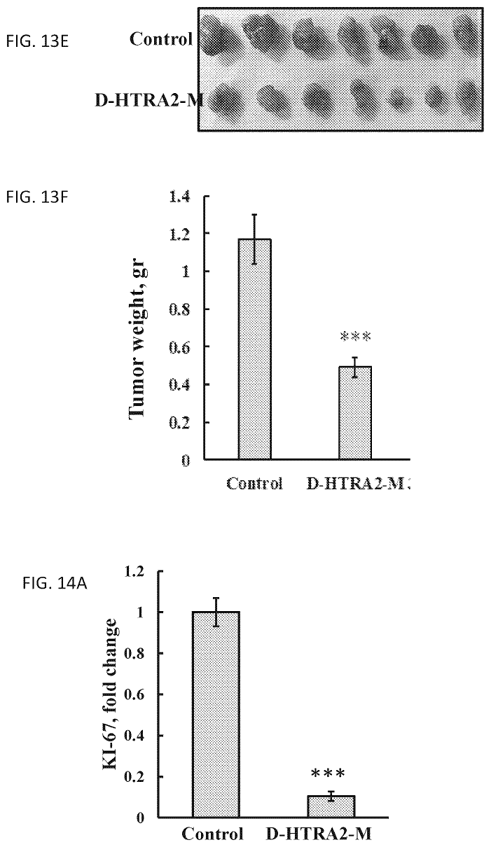

The D-amino acid modified peptide derived from HtrA2/Omi (having SEQ ID NO:35) was used to treat lung cancer in mouse model (FIG. 13). In contrast to tumors not treated with the peptide, which grow exponentially with the tumor volume increasing over 20-fold in 25 days, the volume of tumors treated with the peptide was increased only about 4-fold after 28 days (FIG. 13A). Tumors were photographed (FIG. 13B) and showed up to 80% decrease in their weight upon peptide treatment (FIG. 13C).

Similar results were obtained when the peptides were administrated intravenously (20 mg/kg), showing that HTRA2-All-D-Mito (SEQ ID NO:38) inhibits tumor growth (FIG. 13D-F). Tumor weight showed a decrease of 50% by 1C11-HTRA2-All-D-Mito (FIG. 13F). The results show that the intravenously (IV) administrated peptides reach the subcutaneous tumors and inhibited their growth. The inhibition of tumor growth was lower than when the peptide was administrated intertumorally (IT) (FIG. 12A, 85% IT, compare to 50% LV). However, while in the intratumorally treatment the calculated final concentration of the peptide in the tumor was 50 μM, the peptide concentration in the intravenous treatment although given as 30 mg/Kg, is unknown.

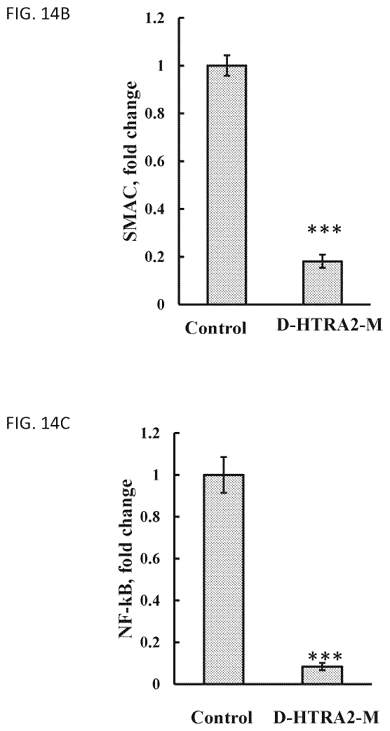

The peptides inhibited tumor growth by inhibiting cell proliferation, as revealed by a decrease of about 90% in the staining of the proliferation marker, Ki-67 of tumor sections (FIG. 14A, B).

Treating the tumors with the 1C11-HTRA2-All-D-Mito peptide also decreased (80%) the expression of SMAC, a protein interacting with PSD and regulating its activity and also, of a key mediator of inflammation, the nuclear transcription factor kappa-B (NF-κB) (Zaidi D, et al., 2018. E. Front Pediatr 6, 317). Immunofluorescence staining of tumor sections with anti-NF-κB antibodies revealed a dramatic decrease (˜90%) in NF-κB expression in the peptide-treated tumors (FIG. 14C). Inflammation is often associated with the development and progression of cancer (Karin M, et al., 2006. Nature 441(7092), 431-436).

### Example 9: PSD and SMAC Overexpressed in Patient-Derived Tumors and Localized Also to the Nucleus

The results presented hereinabove demonstrate that both PSD and SMAC were overexpressed by several fold in cancerous cell lines compared to non-cancerous cells (FIG. 6D, E); thus, the expression of SMAC and PSD was analyzed in sections of patient-derived samples of different cancer types (FIG. 15). Both proteins were overexpressed (3-12-fold) in various tumors including lung, colon, uterus, kidney, pancreas and prostate cancers, relative to their levels in healthy tissues (FIG. 15).

The relative increase in the expression levels of PSD (FIG. 15C) and SMAV (FIG. 15D) in the different patient derived tumors, showed an increase of between 2 and 14 folds in the various tumors.

Interestingly, in lung cancer, both SMAC and PSD were also found in the nucleus (FIG. 15Aa, Bb). This is in agreement with PSD interacting with peptides derived from nuclear proteins such as MAML2, BIRC2, HTRA2, NR4A1, MTFR1, and ARNT (Tables 1, 4).

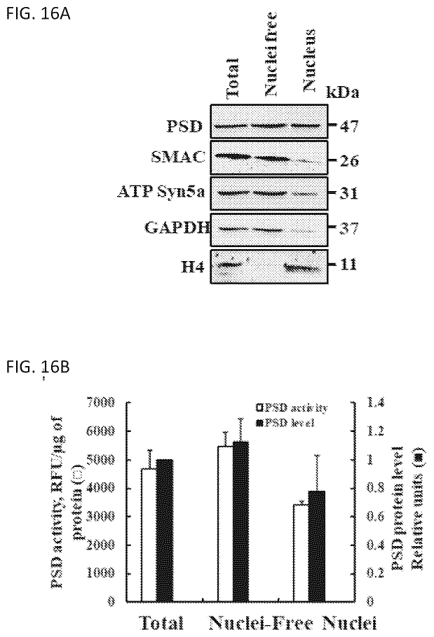

To evaluate the presence of PSD in the nucleus, H1563/CRL-5875 (NCI-H-1563) cells showing the highest expression level of both SMAC and PSD were selected (FIG. 6). Applying nuclear fractionation, PSD is found in the nuclear fraction both at the protein level and protein activity (FIG. 16A, B), and may thus produce PE in the nucleus as proposed previously (Alessenko A V, and Burlakova E B. 2002. Bioelectrochemistry 58, 13-21).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-55. (canceled)

56. An agent capable of modulating the activity of a phosphatidylserine decarboxylase (PSD) within a mammalian cell.

57. The agent according to claim 56, wherein said agent is selected from the group consisting of a peptide, a small molecule, a nucleic acid molecule and any combination thereof.

58. The agent according to claim 56, wherein said agent is capable of at least one of interacting with PSD and inhibiting the activity of PSD.

59. The agent according to claim 58, wherein said agent is a peptide capable of interacting with PSD, wherein the peptide is derived from a polypeptide or a protein interacting with PSD and wherein said peptide comprises L-amino acids, D-amino acids, or a combination thereof.

60. The agent according to claim 59, wherein the polypeptide or protein interacting with PSD is selected from the group consisting of MAML2, HTRA2, BIRC2, TRAF-2, MTFR1, ARNT and any combination thereof.

61. The agent according claim 59, wherein said agent is a peptide comprising an amino acid sequence having at least 80% identity over at least 23 contiguous amino acids of a sequence selected from the group consisting of SEQ ID NOs:1, 4, 5, 2, 6-9, 11-14, an analog, derivative or a fragment thereof.

62. The agent according to claim 59, wherein said agent is a peptide targeted to a cell nucleus or mitochondrion.

63. The agent according to claim 62, wherein the peptide further comprises at least one nuclear or mitochondrial targeting moiety each independently is linked to the N- or the C-terminus of the peptide.

64. The agent according to claim 63, wherein the mitochondrial targeting moiety comprises the amino acid sequence set forth in SEQ ID NO:15 and the nuclear targeting moiety comprises the amino acid sequence set forth in SEQ ID NO:16.

65. The agent according to claim 64, wherein said agent is a peptide comprising an amino acid sequence selected from the group consisting of SEQ ID NO:17, SEQ ID NO:21, SEQ ID NO:25, SEQ ID NO:19, SEQ ID NO:20, SEQ ID NO:24 and SEQ ID NO:28.

66. The agent according to claim 59, wherein said agent is a peptide further comprising a cell penetration moiety enhancing the permeability of the peptide through the plasma membrane of a cell.

67. The agent according to claim 56, wherein the mammalian cell is a human cell.

68. The agent according to claim 56, wherein the cell is a cancerous cell.

69. A pharmaceutical composition comprising at least one agent according to claim 56, further comprising at least one pharmaceutically acceptable diluent, excipient or carrier.

70. A method for treating cancer in a subject in need thereof, comprising administering to the subject a therapeutically effective amount of at least one agent capable of modulating the activity of phosphatidylserine decarboxylase (PSD) or a pharmaceutical composition comprising same.

71. The method according to claim 70, wherein said agent is selected from the group consisting of a peptide, a small molecule, a nucleic acid molecule and any combination thereof.

72. The method of claim 70, wherein the agent is capable of at least one of interacting with PSD and inhibiting the activity of PSD.

73. The method according to claim 72, wherein said agent is a peptide capable of interacting with PSD and wherein said peptide is derived from a polypeptide or a protein interacting with PSD.

74. The method according to claim 70, wherein the agent is capable of inhibiting proliferation of the subject cancer cells.

75. The method according to claim 74, wherein the cancer is selected from the group consisting of lung cancer, breast cancer, colon cancer, esophagus cancer, ovarian cancer, pancreatic cancer, lymphoma, sarcomas, stomach cancer, skin cancer, renal cancer, prostate cancer, testicular cancer, cervical cancer, and leukemia.
